SEQUENCE LISTING (5 sequences)

SEQ ID NO: 1 - RNA, 21 nt, Homo sapiens
aguguucaaucccagaguccc

SEQ ID NO: 2 - DNA, 22 bp, Artificial Sequence
  morpholino of mature miRNA-125b
tcacaagttagggtctcaggga

SEQ ID NO: 3 - DNA, 25 bp, Artificial Sequence
  morpholino of pre-miRNA-125b-1
gtgcacataacaggaaaacgtcaca

SEQ ID NO: 4 - DNA, 25 bp, Artificial Sequence
  morpholino of pre-miRNA-125b-2
cgtggacatcgaagcagaacgtcac

SEQ ID NO: 5 - DNA, 25 bp, Artificial Sequence
  morpholino of pre-miRNA-125b-3
gtgatttttagcacacaaagctcac

(19) United States
(12) Patent Application Publication
(10) Pub. No.: US 2012/0029055 A1
(43) Pub. Date: Feb. 2, 2012

(54) MODULATORS OF APOPTOSIS AND THE USES THEREOF

(75) Inventors: Bing Lim, Singapore (SG); Minh Thi Nguyet, Singapore (SG)

(73) Assignee: Agecy for Science, Technology and Research, Connexis (SG)

(21) Appl. No.: 13/257,228

(22) PCT Filed: Mar. 17, 2010
(86) PCT No.: PCT/SG10/00101

§ 371 Date: Sep. 16, 2011

Related U.S. Application Data
(60) Provisional application No. 61/161,562, filed on Mar. 19, 2009.

(51) Int. Cl. A61K 31/7088; A61K 31/675; A61P 35/00; C07F 9/6558; C12N 15/63; C12N 5/00; C07H 21/02

(52) U.S. Cl. 514/44 A; 435/375; 514/79; 536/24.5; 544/82; 435/320.1; 435/377

(57) ABSTRACT

Here we demonstrate that miR-125b, a brain-enriched microRNA, is a negative regulator of p53 in animals. miR-125b-mediated downregulation of p53 is dependent on the binding of miR-125b to a microRNA response element in the 39 untranslated region of p53 mRNA. Overexpression of miR-125b represses the endogenous level of p53 protein and suppresses apoptosis in cells. In contrast, knockdown of miR-125b elevates the level of p53 protein and induces apoptosis in cells. This phenotype can be rescued significantly by either an ablation of endogenous p53 function or ectopic expression of miR-125b in zebrafish. Ectopic expression of miR-125b suppresses the increase of p53 and stress-induced apoptosis. Together, our study demonstrates that miR-125b is an important negative regulator of p53 and p53-induced apoptosis during development and during the stress response.

### CROSS-REFERENCE TO RELATED APPLICATION

This application claims benefit of, and priority from, U.S. provisional patent application No. 61/161,562, filed on 19 Mar. 2009, the contents of which are hereby incorporated herein by reference.

### FIELD OF TECHNOLOGY

The Technology is in the field of apoptosis regulation useful in cancer or stress treatment or in regenerative medicine.

### BACKGROUND

The p53 tumor suppressor is an important transcription factor that safeguards the cell against tumorigenesis and regulates multiple cellular processes (Foulkes 2007). During development, p53 activity is maintained at a very low level (Almog and Rotter 1997). Activation of p53 in response to DNA damage or oncogene activation leads to cell cycle arrest or apoptosis (Foulkes 2007). Inactivation of the p53 pathway is often a key event in tumorigenesis, found in ˜50% of human cancers (Harris 1996). The expression and activity of p53 are monitored by many layers of regulation, mainly at the post-translational level by ubiquitin ligases such as Mdm2 and Mdm4 (Foulkes 2007). Indeed, changes in the protein level of p53 were not observed to correlate with the transcriptional activity of the p53 gene during embryogenesis or differentiation, indicating that post-transcriptional regulation is likely to be involved (Dony et al. 1985; Klinken et al. 1988; Tchang et al. 1993). Although an antisense RNA is implicated in the maturation of p53 pre-mRNA, there is no evidence that it modulates p53 expression (Khochbin and Lawrence 1989). P53 is an endogenous protein that induces apoptosis of cells. It is generally activated when cells are under stress. The p53 protein known to be inactive in many tumors. More than 50 percent of human tumours contain a mutation or deletion of the TP53 gene.

Micro-RNAs (miRNAs) are an abundant class of small noncoding RNAs, potentially involved in biological processes (Bartel 2004). miRNAs are generated in two steps: First, a hairpin precursor is processed from miRNA primary transcripts by the Drosha complex; and second, the precursor is cleaved by the Dicer complex to become a mature duplex miRNA (Bartel 2004). One strand of the mature miRNA is loaded into the RNA-induced silencing complex (RISC) and brought to its target mRNAs (Bartel 2004). miRNAs often bind to the 39 untranslated region (UTR) of mRNAs with imperfect complementarity (Bartel 2004). They regulate gene expression by inhibiting translation or repressing stability of the targets (Bartel 2004). Inactivation of miRNA biogenesis by the loss of Dicer leads to severe defects in zebrafish embryos and in mouse prenatal lethality (Bernstein et al. 2003; Giraldez et al. 2005). The role of miRNAs during development has also been notably featured in the self-renewal and differentiation of embryonic stem cells and the formation of various embryonic tissues (He and Hannon 2004; Foshay and Gallicano 2007; Tay et al. 2008). The miR-34 family is a key downstream effector of the p53 tumor suppressor. miR-125b, a highly conserved homolog of lin-4 essential for the temporal control of post-embryonic differentiation in worms, it was also recently found to be elevated in several types of cancers and reduced in HCC.

miRNAs are also implicated in tumorigenesis. Differences in miRNA expression profiles define the signatures of various cancers, and miRNA dysregulation can lead to all the hallmarks of cancer (Garzon et al. 2006; Zhang et al. 2007). miRNAs are known to be both regulators and targets of oncogenes and tumor suppressor genes (Garzon et al. 2006). For example, the let-7 miRNA family regulates the Ras oncogenes (Johnson et al. 2005). The miR-34 family is a key downstream effector of the p53 tumor suppressor (Bommer et al. 2007; Chang et al. 2007; He et al. 2007; Tarasov et al. 2007). miR-125b, a highlyconserved homolog of lin-4 essential for the temporal control of post-embryonic differentiation in Caenorhabditis elegans (Olsen and Ambros 1999), was also recently found to be elevated in several types of cancers (Nelson et al. 2006; Bloomston et al. 2007; Shi et al. 2007; Bousquet et al. 2008). Further understanding of the expression and the function of miRNAs in cancers would provide new approaches for diagnosis and therapies against these diseases.

Recent reports suggest that expression of miR-125b is down-regulated in ovarian carcinoma and thyroid carcinoma (Iorio et al. 2007; Nelson et al. 2006; Volinia et al. 2006; Nam et al. 2008 Le et al. 2008) but elevated in pancreatic cancer, oligodendroglial tumors, prostate cancer, myelodysplastic syndromes (MDS), and acute myeloid leukemia (AML) (Nelson et al. 2006; Bloomston et al. 2007; Shi et al. 2007; Bousquet et al. 2008). Particularly in MDS and AML patients, a t(2:11)(p21:q23) translocation with the breakpoint mapped near the genomic location of mir-125b-1 locus leads to a sixfold to 90-fold up-regulation of miR-125b (Bousquet et al. 2008). In addition, miR-125b was shown to suppress cell cycling in hepatocellular carcinomas (Li et al. 2008) but to promote proliferation of prostate cancer cells (Shi et al. 2007). The biological function of miR-125b is currently unclear and what is known suggest very contradictories roles requiring clarity.

Morpholinos are a useful tool for loss-of-function studies in zebrafish; however, their frequent off-target effect is a main concern for functional analysis (Ekker and Larson 2001). Particularly, increase in neural cell death by 24 hpf has been considered a nonspecific effect of 15%-20% of the morpholinos used in zebrafish (Ekker and Larson 2001). Recently, Robu et al. (2007) demonstrated that this effect is mediated through the p53 pathway since the increase in apoptosis is associated with p53 activation and can be completely reversed by coinjection of p53 morpholino. The specific mechanism by which p53 is activated by mistargeting of morpholinos was not explained (Robu et al. 2007). The question is whether activation of p53 and increase in neural cell death is always an off-target effect. There are many examples showing that the overexpression of p53 or knockdown of p53's negative regulators can lead to the same phenotype in zebrafish (Langheinrich et al. 2002; Campbell et al. 2006; Ghiselli 2006; Bretaud et al. 2007). Robu et al. (2007) suggested that even if the morpholino targets a known regulator of p53, it can also have some off-target effect, as in the case of a Mdm2 morpholino. They believe that a morphant phenotype can be considered as on-target only if it can be rescued by overexpression of the targeted gene (Robu et al. 2007). This approach has been used previously to identify new regulators of p53 in zebrafish (Ghiselli 2006).

Cancer is one of the main diseases of the 21^{st} century causing 13% of all deaths. New aspects of the genetics of cancer pathogenesis, such as DNA methylation and microRNAs are increasingly recognized as important. While there are several chemical that can effect rapidly dividing cancer cells most of these are toxic with adverse side effects. Artificially elevating levels of p53 has been shown to cause premature ageing. Restoring endogenous p53 induced apoptosis in tumour cells is desirable in treating cancer but there are no such methods on the market.

### SUMMARY

Modulation of MicroRNA-125b has a direct effect on p53 activity and consequently apoptosis. Inhibiting MiR-125b induces apoptosis through increased endogenous p53 activity. Upregulation of MicroRNA-125b inhibits endogenous p53 activity reducing apoptosis.

Accordingly, one aspect of the invention includes a method of regulating apoptosis comprising modulation of the level of a micro RNA-125b having a microRNA response element (MRE) comprising SEQ ID NO. 1, by administering a modulator to the cell.

In one embodiment the method is in vitro. In another embodiment the method is in vivo, and the modulator is administered to a subject.

In one embodiment apoptosis is upregulated by inhibiting the level of the micro RNA-125b in the cell, by administering the modulator to the cell. Such a method may be effective in cancer treatment where the inhibitor is administered to a subject in need of cancer treatment.

The inhibitor may be a microRNA-125b antisense oligonucleotide. Alternatively, the inhibitor may be a microRNA-125b morpholino.

In one embodiment apoptosis is downregulated by enhancing the level of the micro RNA-125b in the cell, by administering the modulator to the cell. Such a method may be effective in cell differentiation where the agonist is administered to an undifferentiated cell to enhance cell differentiation

The agonist may comprise a vector incorporating a micro RNA-125b having a microRNA response element (MRE) comprising SEQ ID NO. 1 Alternatively, the agonist may comprise a microRNA-125b duplex molecule.

Another aspect of the invention provides a composition for use in regulating apoptosis in a cell wherein the composition modulates the level of a micro RNA-125b having a microRNA response element (MRE) comprising SEQ ID NO. 1.

In one embodiment the composition comprises an inhibitor of micro RNA-125b, inhibiting the level of the micro RNA-125b in the cell, wherein apoptosis is upregulated by the inhibitor.

The inhibitor may be a microRNA-125b antisense oligonucleotide. Alternatively, the inhibitor may be a microRNA-125b morpholino.

In another embodiment the composition comprises an agonist of micro RNA-125b, enhancing the level of the micro RNA-125b in the cell, wherein apoptosis is downregulated by the agonist.

The agonist may comprise a vector incorporating a micro RNA-125b having a microRNA response element (MRE) comprising SEQ ID NO. 1 Alternatively, the agonist may comprise a microRNA-125b duplex molecule.

### DESCRIPTION OF THE DRAWINGS

FIG. 1: miR-125b binds to the 3′ UTR of zebrafish and human p53 mRNAs. (A) Outline of luciferase reporter assay for validating the interaction of miR-125b with the 3′ UTR of p53: The MREs of miR-125b in the 3′ UTR of human and zebrafish p53 mRNA were predicted by TargetScan and miRBase Target. Shaded texts indicate the “seed” regions. Each predicted MRE or the whole p53 3′ UTR was inserted into a psiCheck2 vector, immediately downstream from the Renilla luciferase gene. In mutant reporter constructs, the MRE was deleted or a three-mismatch mutation was introduced into the seed region. Each luciferase construct was co-transfected with negative control duplex 1 (NC-DP1) or miR-125b duplex (125b-DP) into HEK-293T cells, and luciferase readings were obtained 48 h after transfection. (B) Repression of luciferase activity due to the interaction between miR-125b and the predicted MREs in the luciferase-MRE or in the luciferase-p53-39 UTR constructs. Repression was abolished when the MRE was deleted or mutated. Every Renilla luciferase reading was normalized to that of the control firefly luciferase. The luciferase activities of 125b-DP-transfected cells were presented as percentages relative to the level of luciferase in the NC-DP1-transfected cells (this control luciferase level is considered as 100% and is represented by the solid red line). The values represent average±SEM (n≧6). The dashed line represents the threshold of luciferase activity (75%), suppression of luciferase level below which indicates positive binding. Two-tail t-test results are indicated by (*) P<0.05 and (**) P<0.01, relative to the NC-DP1-transfected controls. (C) Overexpression of miR-125b down-regulates the wild-type but not the MRE-deleted human p53 (hp53) in H1299 cells: pcDNA3.1⁺ vector containing the full-length human p53 cDNA sequence with or without the miR-125b MRE was transfected alone or co-transfected with negative control duplex 3 (NC-DP3) or 125b-DP into H1299 cells. The level of human p53 was analyzed by Western blots 2 d after transfection. (D) Overexpression of miR-125b downregulates the wild-type but not the MRE-deleted zebrafish p53 (fp53) in H1299 cells: pcDNA3.1⁺ vector containing the full-length zebrafish p53 cDNA sequence with or without the MRE of miR-125b was transfected alone or co-transfected with NC-DP3 or 125b-DP into H1299 cells. The level of zebrafish p53 was analyzed by Western blots 2 d after transfection. (E) p53 protein level was quantified from the Western blot bands in C and D, normalized to GAPDH level, and presented as fold change±SEM (n≧3) relative to the p53 level of p53-only-transfected cells (solid red line). The dashed line represents the threshold of suppression (0.75-fold) corresponding to threshold set in the luciferase reporter assay. Two-tail t-test results are indicated by (*) P<0.05 and (**) P<0.01, relative to the p53-only-transfected controls.

FIG. 2: miR-125b represses the endogenous p53 expression and suppresses p53-induced apoptosis in human neuroblastoma SH-SY5Y cells. (A) The endogenous p53 protein level in SHSY5Y cells 2 d after a transfection with mock (water), negative control duplex 3 (NC-DP3), miR-125b duplex (125b-DP), or p53 siRNA. (B) The p53 protein level was quantified from the Western blot bands in A, normalized to the GAPDH level, and presented as fold change±SEM (n≧3) relative to the p53 level of mock-transfected cells. (C) The mRNA expression levels of p53, p21, and bax in SH-SY5Y cells 2 d after transfection with NC-DP1 or 125b-DP. The expression was quantified by real-time PCR, normalized to the expression of β-actin, and presented as fold change±SEM (n≧4) relative to that in the cells transfected with NC-DP1. (D) The percentage of SH-SY5Y cells positive for active caspase-3 was quantified by the Cellomics high-content screening system 2 d after a transfection with NCDP1 or with 125b-DP. The 10 mM H-7 treatment was applied 24 h before fixing. The values represent average±SEM (n≧3). For each replicate, 20 images (including at least 10,000 cells) were analyzed. In all panels, two-tail t-test results are indicated by (*) P<0.05 and (**) P<0.01, relative to the mock-transfected or NC-DP-transfected controls.

FIG. 3: miR-125b represses the endogenous p53 expression and suppresses apoptosis in human lung fibroblast cells. (A) The endogenous p53 level in human lung fibroblast cells 2 d after transfection with mock (water), negative control duplex 2 (NC-DP2), or miR-125b duplex (125b-DP); and 1 d after transfection with mock, negative control antisense 1 (NC-AS1), or miR-125b antisense (125b-AS). (B) The p53 protein level was quantified from the Western blot bands in A, normalized to the GAPDH level and presented as fold change±SEM (n≧3) relative to the p53 level of mock-transfected cells (dotted line). (C) The levels of p53 mRNA and p21 mRNA in human lung fibroblast cells 2 d after transfection with mock, NC-DP2, 125b-DP, or NC-AS1, 125b-AS, or a co-transfection of 125b-AS and p53 siRNA. The expression was quantified by real-time PCR, normalized to the expression of β-actin and presented as fold change SEM (n≧4) relative to that in the mock-transfected cells (dotted line). (D) The percentage of human lung fibroblast cells positive for active caspase-3, 2 d after transfection with mock, NC-DP2, 125b-DP, NC-AS1, or 125b-AS was quantified by the Cellomics high-content screening system. The values represent average±SEM (n≧3). For each replicate, 20 images (including at least 10,000 cells) were analyzed. In all panels, two-tail t-test results are indicated by (*) P<0.05 and (**) P<0.01, relative to the mock-transfected controls.

FIG. 4: Spatio-temporal expression of miR-125b during zebrafish embryogenesis. (A) Whole-mount in situ hybridization of miR-125b in zebrafish embryos at 19 hpf, 22 hpf, 26 hpf, 30 hpf, and 48 hpf. Side view of the whole body excluding the tail is shown. (B) Side view of zebrafish brain, in situ hybridization with miR-125b at 22 hpf, 26 hpf, 30 hpf, and 48 hpf. In A and B, each image shows the expression pattern of miR-125b in a representative embryo. The same pattern was observed in all 20 embryos examined at each developmental stage. (ey) Eye; (hb) hindbrain; (hyp) hypothalamus; (mhb) midbrain-hindbrain boundary; (ot) optic tectum; (tel) telencephalon; (tg) tegmentum. (C) The expression pattern of miR-125b, p53, and p21 during zebrafish development: Transcript levels were quantified by real-time PCR, normalized to internal controls (18S or β-actin), and presented as log₂ fold change±SEM (n≧4) relative to the expression at 18 hpf.

FIG. 5: Loss of miR-125b in zebrafish embryos. (A) Design of morpholinos targeting either the guide strand of mature miR-125b (m125b) or the loop regions of pre-mir-125b (lp125b). Three different lp125b morpholinos (lp125bMO1/2/3) were designed for the three isoforms of pre-mir-125b. (B) qRT-PCR elucidating the effects of miR-125b morpholinos on the endogenous level of zebrafish miR-125b at 24 hpf. One-cell-stage embryos were injected with m125bMO or lp125bMO1/2/3 (individually or together). A morpholino (misMO) with 5 nt different from m125bMO was used as control. Total RNA was obtained from the embryos at 24 hpf. All the expression values were normalized to 18S RNA levels and presented as average percentage±SEM (n≧4) relative to the expression values in uninjected controls. Two-tail t-test results are indicated by (**) P<0.01, relative to the uninjected control. (C) Loss-of-function morphology at 24 hpf: Morphants typically exhibit severe cell death in the brain (brackets), absence of the midbrain-hindbrain boundary (*), smaller eyes (blue arrows), and deformed somites (green arrows). Each control/morphant embryo is shown with a lateral view of the whole body and a magnified view of the head. The total number of embryos (n) in each treatment and the percentage of embryos having the same phenotype as in the representative picture are indicated below each image.

FIG. 6: Loss of miR-125b elevates p53 and triggers p53-dependent apoptosis in zebrafish embryos. (A) Elevation of p53 protein caused by loss of miR-125b in zebrafish embryos: Embryos were injected with misMO, m125bMO, or lp125bMO1/2/3. Western blotting was performed at 24 hpf. (B) The p53 protein level was quantified from the Western blot bands in A, normalized to tubulin level, and presented as fold change±SEM (n≧3) relative to the p53 level in the misMO-injected embryos. Two-tail t-test results are indicated by (**) P<0.01, relative to the misMO-injected control. (C) qRT-PCR of p21 transcripts at 24 hpf in embryos injected with different combinations of morpholinos. p53MO indicates a morpholino blocking translation of p53. The values were normalized to the expression level of β-actin and represented as average fold change±SEM (n≧4) relative to the expression level in misMO-injected embryos (dashed line). Two-tail t-test results are indicated as (**) P<0.01. (D) TUNEL assay for detecting apoptotic cells (visualized as red spots) in the 24-hpf brains and acetylated tubulin staining (aAT) marking mature neurons and axonal tracts in the 48-hpf brains of wild-type and p53M214K mutant embryos microinjected with misMO, m125bMO, or lp125bMO1/2/3. Each image is a projection of multiple optical slides obtained from a representative embryo. Three embryos were observed for each condition for the TUNEL assay, and five were observed for each condition in the aAT staining. All of them had a similar phenotype as the representative images. (AC) Anterior commissure; (d) diencephalon; (fb) forebrain; (hb) hindbrain; (mb) midbrain; (MLF) medial longitudinal fasciculus; (ot) optic tectum; (SOT) supraoptic tract; (TPC) tract of posterior commissure; (TPOC) tract of postoptic commissure; (t) telencephalon. Bar, 50 mm.

FIG. 7: Synthetic miR-125b rescues apoptosis in miR-125b morphants. (A) TUNEL assay for detecting apoptotic cells in the 24-hpf brains: Embryos were injected with a standard negative control morpholino, miR-125b duplex (125b-DP), m125bMO, or lp125bMO1/2/3. Two different concentrations of 125b-DP (12.5 fmol and 37.5 fmol per injection) were used to rescue the embryos injected with lp125bMO1/2/3. Each image is a projection of multiple optical slides from a representative embryo. Three embryos were observed for each condition, and all of them had a similar phenotype as the representative images. (fb) Forebrain; (hb) hindbrain; (mb) midbrain. Bar, 50 mm. (B) Regulation of p53 protein in the morphants and the rescued embryos. Western blotting was performed at 24 hpf. (C) p53 protein level was quantified from the Western blot bands in B, normalized to tubulin level, and presented as fold change±SEM (n≧3) relative to the p53 level in the embryos injected with misMO. Two-tail t-test results are indicated by (**) P<0.01.

FIG. 8: Overexpression of miR-125b rescues stress-induced apoptosis. (A) Regulation of p53 protein in zebrafish embryos injected with negative control duplex 1 (NC-DP1), p53 morpholino (p53 MO), or miR-125b duplex (125b-DP). At 24 hpf, uninjected and injected embryos were treated with 500 nM camptothecin for 8 h or subjected to 40 Gy of γ-irradiation. Protein lysate from the two treatments with two sets of untreated control were loaded on two separate gels. The bar chart presents quantification of p53 Western blot band intensity, normalized to tubulin levels, and presented as fold change relative to the uninjected untreated control of each blot. (B) Regulation of miR-125b in uninjected embryos or those treated with 500 nM camptothecin for 8 h or subjected to 40Gy of γ-irradiation, normalized to 18S RNA level, and presented as average fold change relative to untreated control d SEM (n≧6). Two-tail t-test results are indicated as (**) P<0.01, relative to the untreated control. (C) Staining of apoptotic cells in embryos uninjected or injected with NC-DP1, p53 MO, or 125b-DP, treated with 500 nM camptothecin for 8 h or with 40 Gy of γ-irradiation. Embryos were fixed at 32 hpf and subjected to TUNEL assay. Each image is a projection of multiple optical slides from a representative embryo. Three embryos were observed for each condition, and all of them had a similar phenotype as in the representative image. (fb) Forebrain; (hb) hindbrain; (mb) midbrain. Bar, 50 mm.

FIG. 9: (A) Predicted miRNA response elements (MREs) of miR-125b in the p53 mRNAs of monkey, rabbit, horse and frog. Shaded text indicates the seed regions. (B) Luciferase reporter assay validating the specificity and efficacy of miR-125b duplex and antisense. 293T cells were transfected with a Renilla luciferase reporter construct containing a sequence perfectly complementary to human miR-125b. The cells were co-transfected with negative control duplex 1/2/3 (NC-DP1/2/3) or miR-125b duplex (125b-DP) at a final concentration of 10 nM; negative control antisense 1 (NC-AS1) or miR-125b antisense (125b-AS) at a final concentration of 100 nM. The assay was performed two days after transfection. Every Renilla luciferase reading was normalized to that of the control firefly luciferase. The luciferase activity is presented as percentages relative to the level of luciferase in NC-DP1 transfected cells (dashed line). The values represent average±s.e.m. (n≧6). (C) The level of miR-125b in H1299 cells two days after a transfection with NC-DP3 or 125b-DP and plasmids expressing human wild-type p53 or human MRE-deleted p53. The level of miR-125b was quantified by real-time PCR, normalized to the expression of U6 RNA and presented as log₂ fold change±s.e.m. (n≧4) relative to that in the p53-only-transfected cells. Two-tail T-test results are indicated by *P<0.05 and ** P<0.01.

FIG. 10: Validation of miR-125b overexpression and knockdown in SHSY5Y cells and in human lung fibroblast cells (A) The level of miR-125b in SH-SY5Y cells two days after a transfection with negative control duplex 3 (NC-DP3) or miR-125b duplex (125b-DP). (B) The level of miR-125b in human lung fibroblast cells two days after a transfection with NCDP2, 125b-DP, negative control antisense 1 (NC-AS1) or miR-125b antisense (125b-AS). For both (A) and (B), the level of miR-125b was quantified by real-time PCR, normalized to the expression of U6 RNA and presented as log₂ fold change±s.e.m. (n≧4) relative to miR-125b level in the mock-transfected cells. Two-tail T-test results are indicated by ** P<0.01, relative to the mock-transfected control

FIG. 11: (A) Real-time PCR analysis of p21 mRNA levels in human lung fibroblasts transfected with miR-125b duplex (125b-DP) at different concentrations. The level of p21 mRNA was normalized to the expression of β-actin and presented as fold change±s.e.m. (n≧4) relative to the p21 level in mock transfected cells. Two-tail T-test results are indicated by * P<0.05 and ** P<0.01. (B) Western blot analysis of p53 protein in SH-SY5Y cells treated with 10 μM etoposide or with DMSO vehicle control for 24 hours. Tubulin was used as a loading control. (C) Real-time PCR analysis of miR-125b expression level in SH-SY5Y cells treated as in (B). The level of miR-125b was normalized to the expression of U6 RNA and presented as fold change s.e.m. (n≧4) relative to miR-125b level in DMSO treated cells. Two-tail T-test results are indicated by ** P<0.01.

FIG. 12 (A) The level of miR-125b in Swiss-3T3 cells two days after a transfection with miR-125b duplex (125b-DP). The level of miR-125b was quantified by real-time PCR, normalized to the expression of U6 RNA and presented as log₂ fold change±s.e.m. (n≧4) relative to miR-125b level in the mock-transfected cells. (B) The mRNA levels of p53, p21 and bax in Swiss-3T3 cells two days after a transfection with mock or 125b-DP. The expression was quantified by real-time PCR, normalized to the expression of β-actin and presented as fold change±s.e.m. (n≧4) relative to that in the mocktransfected cells. (C) Real-time PCR analysis of seven miR-125b putative targets in the p53 pathway. The expression of seven genes was measured in human SH-SY5Y cells transfected with negative control duplex 1 (NC-DP1) or 125b-DP or in mouse Swiss-3T3 cells transfected with mock or 125b-DP. The level of each mRNA was normalized to the expression of β-actin and presented as fold change±s.e.m. (n≧4) relative to the mock/NC-DP1. Two-tail T-test results are indicated by * P<0.05 and ** P<0.01. The expression of PLAGL1 was not detected in Swiss-3T3 cells.

FIG. 13: Developmental onset of apoptosis in miR-125b morphants: Embryos were injected with either misMO or m125bMO. Cell death was observed in the brain by live imaging of the head and fluorescent imaging of fixed embryos where apoptotic cells were stained by a TUNEL assay. Each fluorescent image is a projection of multiple optical slides from a representative embryo. Three embryos were observed in each condition and all of them have a similar phenotype as in the representative image. Scale bar, 50 μm.

FIG. 14: Rescue of miR-125b morphants by the loss of p53. The phenotype of miR-125b morphants were reversed by co-injection of miR-125b morpholinos with p53 morpholino, or by injecting miR-125b morpholinos into p53M214K homozygous mutants. Images were captured at 24 hpf. Morphants typically exhibit severe cell death in the brain (brackets) and absence of midbrain-hindbrain boundary (*), smaller eyes (blue arrows) and deformed somites (green arrows). Each control/morphant embryo is shown with a lateral view of the whole body and a magnified view of the head. The total number of embryos (n) in each treatment and the percentage of embryos having the same phenotype as in the representative embryo are indicated below each image.

### DETAILED DESCRIPTION

Here, we report for the first time a miRNA that directly regulates p53. We identified microRNA-125b (miR-125b) as a negative regulator of p53 in both humans and zebrafish. miR-125b binds directly to the 3′ UTR of human and zebrafish p53 mRNAs, and represses p53 protein levels in a manner dependent on its binding site in the p53 3′ UTRs. miR-125b-mediated regulation of p53 is critical for modulating apoptosis in human cells and in zebrafish embryos during development and during the stress response. Our report provides new insights into the function of miR-125b during development and suggests a role in tumorigenesis for this miRNA.

We showed that microRNA-125b suppress p53. We also have evidence for that miR-125b suppress other genes in the p53 pathway. Therefore miR-125b is an important regulator of p53 levels and p53 pathways in cells. It is known that loss of p53 predispose cells to survive stress and DNA-damage induced death and selection for cancer cells. Many cancers have reduced p53 or loss of p53. In these instances, p53 pathway would be suppressed. Therefore a way to restore p53 function would be to knockdown levels of microRNA 125b to allow p53 protein level in tumor cells to increase.

The technology then involves the use of inhibitors of microRNA-125b to Induce death of cancer cells. Our claim is based on a specific link between a specific microRNA and a key tumor suppressor gene, p53 that mediates carcinogenesis in many types of human cancers.

MicroRNA are small molecules. Antimirs and various inhibitors of microRNAS being developed are also small molecules. Therefore microRNAs and inhibitors of microRNAs can be used directly as drug.

Accordingly a method of regulating apoptosis is described the method comprising modulation of the level of a micro RNA-125b having a microRNA response element (MRE) comprising SEQ ID NO. 1, by administering a modulator to the cell. In one embodiment the method is in vitro. In another embodiment the method is in vivo, and the modulator is administered to a subject. The subject may be from any of the species listed in table 2. Preferably the subject is a mammal and most preferably a human.

“Micro RNA-125b” as used herein also known as miR-125b, or miRNA-125b is a small ribonucleic acid including within a section of its sequence a microRNA response element (MRE) comprising SEQ ID NO. 1.

### Modulators of Micro RNA-125b

A substance that modulates the level of a micro RNA-125b having a microRNA response element (MRE) comprising SEQ ID NO. 1 in a cell may do so in several ways. It may directly disrupt the miR-125b and block its activity. Candidate substances of this type may conveniently be preliminarily screened by in vitro binding assays as, for example, described below and then tested, for example in a whole cell assay as described below. Examples of candidate substances include antisense RNA or double-stranded interfering RNA sequences such as miR-125b antisense or a morpholino of a mature miR-125b such as that disclosed in SEQ ID. NO.: 2.

A substance which can bind directly to miR-125b may also inhibit the level of a micro RNA-125b in a cell. This can be tested using, for example the whole cells assays described below. In particular, fragments of substance which can bind directly to miR-125b which comprise microRNA response element (MRE) of SEQ ID NO. 1 may be suitable to inhibit the level of a micro RNA-125b in a cell.

Where modulating the level of a micro RNA-125b in a cell comprises inhibiting or reducing the level of a micro RNA-125b in a cell the modulator/inhibitor may comprise a microRNA-125b antisense oligonucleotide. Alternatively, the inhibitor may comprise a microRNA-125b morpholino such as the morpholino of SEQ ID. NO.: 2, or the morpholino of SEQ ID. NO.: 3, or the morpholino of SEQ ID. NO.: 4, or the morpholino of SEQ ID. NO.: 5, or one or more morpholino selected from SEQ ID. NO.: 2; SEQ ID. NO.: 3; SEQ ID. NO.: 4 and SEQ ID. NO.: 5.

Alternatively, instead of inhibiting the level of a micro RNA-125b in a cell the modulator substance may enhance the level of a micro RNA-125b in a cell. This may be by exogenous expression of the miR-125b, for example suitable candidate substances include a vector expressing miR-125b. Preferably the vector may incorporate a micro RNA-125b having a microRNA response element (MRE) including SEQ ID NO. 1 The agonist may comprise a microRNA-125b duplex molecule.

Suitable candidate substances also include antibody products (for example, monoclonal and polyclonal antibodies, single chain antibodies, chimeric antibodies and CDR-grafted antibodies) which are specific for miR-125b. Furthermore, combinatorial libraries, peptide and peptide mimetics, defined chemical entities, oligonucleotides, and natural product libraries may be screened for activity as regulators of miR-125b levels in a cell. The candidate substances may be used in an initial screen in batches of, for example 10 substances per reaction, and the substances of those batches which are inhibitory or agonistic, tested individually. Candidate substances which show activity in in vitro screens such as those described below can then be tested in whole cell systems, such as mammalian cells or in zebrafish development, which will be exposed to the inhibitor or agonist and tested for effects on cell apoptosis.

### Nucleic Acid Constructs and Vectors

Polynucleotides of the invention may be incorporated into a recombinant replicable vector for introduction into a prokaryotic or eucaryotic host. Such vectors may typically comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and RNA stabilizing sequences. Further fusion proteins capable of regulating dimerization such as FKBP may be included in a plasmid to facilitate localized dimerization in the presence of rapamycin or FK506 or any other immunosuppressive drugs that naturally act as dimerizers of FKBP and mTOR. Such vectors may be prepared by means of standard recombinant techniques well known in the art.

An appropriate promoter and other necessary vector sequences will be selected so as to be functional in the host, and may include, when appropriate, those naturally associated with miRNA-125b. Examples of workable combinations of cell lines and expression vectors are described in Sambrook et al., 1989. Many useful vectors are known in the art and may be obtained from such vendors as Stratagene, New England Biolabs, Promega Biotech, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in Hitzeman et al., EP 73,675A. Appropriate non-native mammalian promoters might include the early and late promoters from SV40 or promoters derived from murine Moloney leukemia virus, mouse tumour virus, avian sarcoma viruses, adenovirus II, bovine papilloma virus or polyoma. In addition, the construct may be joined to an amplifiable gene (e.g., DHFR) so that multiple copies of the gene may be made. For appropriate enhancer and other expression control sequences.

While such expression vectors may replicate autonomously, they may also replicate by being inserted into the genome of the host cell, by methods well known in the art.

Expression and cloning vectors will likely contain a selectable marker, further it may contain a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting microRNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the microRNA polynucleotides into the host cell by any method known in the art, including, inter alia, those described above, will be referred to herein as “transformation.” The cells into which have been introduced nucleic acids described above are meant to also include the progeny of such cells.

Thus the present invention provides host cells transformed or transfected with a nucleic acid molecule of the invention. Preferred host cells include bacteria, yeast, mammalian cells, plant cells, insect cells, and human cells in tissue culture. Illustratively, such host cells are selected from the group consisting of E. coli, Pseudomonas, Bacillus, Streptomyces, yeast, CHO, R1.1, B-W, L-M, COS 1. COS 7, BSC1, BSC40, BMT10, human HEK-293T cells, human neuroblastoma SH-SY5Y cells, p53-null human lung carcinoma H1299 cells, mouse Swiss-3T3 cells, human lung fibroblast cells and Sf9 cells.

Large quantities of the microRNA of the present invention may be prepared by expressing the microRNA-125b nucleic acids or portions thereof in vectors or other expression vehicles in compatible prokaryotic or eucaryotic host cells. The most commonly used prokaryotic hosts are strains of Escherichia coli, although other prokaryotes, such as Bacillus subtilis or Pseudomonas may also be used.

Thus, the present invention also provides methods for preparing an microRNA-125b comprising: (a) culturing a cell as described above under conditions that provide for expression of the microRNA-125b; and (b) recovering the expressed microRNA-125b. This procedure can also be accompanied by the steps of: (c) chromatographing the microRNA-125b using any suitable means known in the art; and (d) purifying the microRNA-125b by for example filtration methods known in the art.

Method for Treating a Patient with Cancer

On the basis of the above, the present invention provides a method for treating a patient with cancer, which comprises the step of: contacting the cells within and around a cancer with an inhibitor of miR-125b as described above. Desirably, the inhibitor is provided in a therapeutic effective amount.

An alternative form of the present invention resides in the use of the peptide in the manufacture of a medicament for treating a patient with cancer preferably a medicament used in treatment to induce endogenous p53 expression by inhibiting the level of miR-125b in cells within and around a cancer.

“Treatment” and “treat” and synonyms thereof refer to both therapeutic treatment and prophylactic or preventative measures, wherein the object is to prevent or slow down (lessen) a cancer condition. Those in need of such treatment include those already diagnosed with cancer.

As used herein a “therapeutically effective amount” of a compound will be an amount of active miR-125b inhibitor that is capable of preventing or at least slowing down (lessening) a cancer condition, in particular increasing the average 5 year survival rate of cancer patients. Dosages and administration of an antagonist of the invention in a pharmaceutical composition may be determined by one of ordinary skill in the art of clinical pharmacology or pharmacokinetics. An effective amount of the miR-125b inhibitor to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μg/kg/day to 10 mg/kg/day.

### Compositions of the Invention

modulators produced according to the invention can be administered for the treatment of cancer in the form of pharmaceutical compositions.

Thus, the present invention also relates to compositions including pharmaceutical compositions comprising a therapeutically effective amount of a modulator of miR-125b. As used herein a compound inhibitor will be therapeutically effective if it is able to affect cancer growth either in vitro or in vivo. Alternatively, a compound agonist will be therapeutically effective if it is able to affect cell differentiation or development either in vitro or in vivo.

Pharmaceutical forms of the invention suitable for injectable use include sterile aqueous solutions (where water soluble) or dispersions and sterile powders for the extemporaneous preparation of sterile injectable solutions and or one or more carrier. Alternatively, injectable solutions may be delivered encapsulated in liposomes to assist their transport across cell membrane. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active peptides in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

When the active ingredients, in particular small biomolecules contemplated within the scope of the invention, are suitably protected they may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active peptide in such therapeutically useful compositions in such that a suitable dosage will be obtained. Preferred compositions or preparations according to the present invention are prepared so that a dosage unit form contains between about 0.1 μg and 20 g of active compound.

The tablets, troches, pills, capsules and the like may also contain binding agents, such as, for example, gum, acacia, corn starch or gelatin. They may also contain an excipient, such as, for example, dicalcium phosphate. They may also contain a disintegrating agent such as, for example, corn starch, potato starch, alginic acid and the like. They may also contain a lubricant such as, for example, magnesium stearate. They may also contain a sweetening agent such a sucrose, lactose or saccharin. They may also contain a flavouring agent such as, for example, peppermint, oil of wintergreen, or cherry flavouring.

When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparaben as preservatives, a dye and flavouring such as, for example, cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents may also include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

Supplementary active ingredients can also be incorporated into the compositions. Preferably those supplementary active ingredients are anticancer agents such as chemotherapy agents like, for example; cisplatin, platinum or carboplatin in combination with one or more of gemcitabine, paclitaxel, docetaxel, etoposide, vinorelbine, topotecan, or irinotecan; tyrosine kinase inhibitors (e.g., Axitinib, Bosutinib, Cediranib, Dasatinib, Erlotinib, Gefitinib, Imatinib, Lapatinib, Lastaurtinib, Nilotinib, semaxanib, siinitinib, vandetanib, vatalanib or any other suitable tyrosine kinase inhibitor); apoptosis inducing enzymes, for example TNF polypeptides, TRAIL (TRAIL R1, TRAIL R2) or FasL, Exisulind or other apoptosis inducing enzymes; micro-RNA that initiates apoptosis; or other chemotherapy agents such as those commonly known to a person skilled in the art. Alternatively they may be anticancer treatments such as radiotherapy, chest radiotherapy, surgical resection, the chemotherapy agents mentioned above or any combination of these.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 pg to about 2000 mg/ml of carrier. In the case of compositions containing supplementary active ingredients, the dosages are determined by reference to the usual dose and manner of administration of the said ingredients.

The compositions may be for use in treating cancer. Use includes use of a composition of the invention for the preparation of a medicament or a pharmaceutically acceptable composition for the treatment of cancer. The preparation may further comprise a chemotherapeutic agent for the preparation of a medicament for the treatment of cancer

### Preferred Embodiments

The function of miR-125b in Regulating p53 and p53-Dependent-Apoptosis

Our data establish miR-125b as a bona fide negative regulator of p53. We validated this interaction in two vertebrate species, humans and zebrafish, suggesting that this interaction is an essential negative regulatory element of the p53 pathway. The direct interaction between miR-125b and p53 mRNA was elucidated by several lines of evidence: (1) The 3′ UTR of both human and zebrafish mRNAs contain a putative binding site (the MRE) for miR-125b with significant seed match. (2) miR-125b suppresses the activity of a luciferase reporter fused with the 3′ UTR of human/zebrafish p53 mRNA in a MRE dependent manner. (3) miR-125b represses the ectopic expression of human/zebrafish wild-type p53 cDNA but not the MRE-deleted p53 cDNA in a p53-null background (H1299 cells). Our report is the first to identify a miRNA that directly regulates p53.

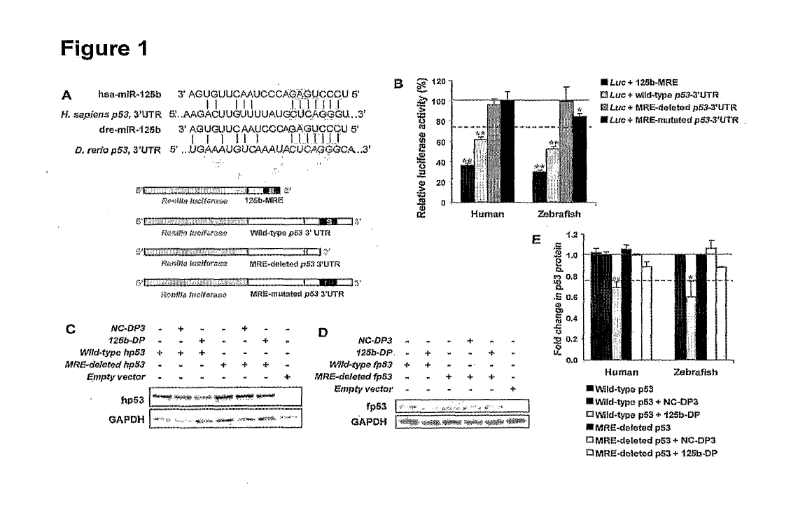

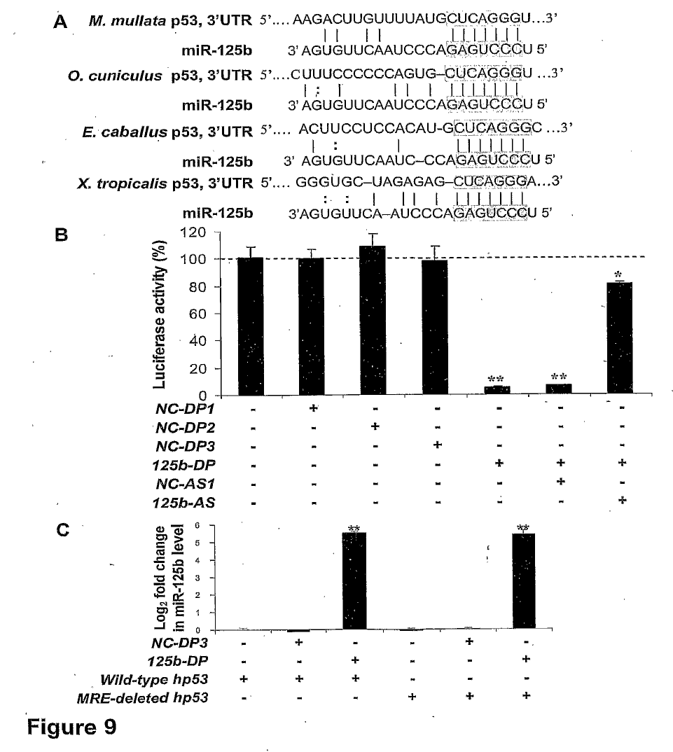

To examine the possibility of p53 regulation by miRNAs, we searched for potential miRNA-binding sites in the p53 mRNA by computational analysis. Searches by TargetScan (Lewis et al. 2005) and miRBase Target (Yoon and De 2006) yielded two different lists of miRNAs. Most of the predicted binding sites (miRNA response elements, MREs) of these miRNAs in the 3′ UTR of p53 are poorly conserved. Only one specific miRNA, miR-125b, targeted both human and zebrafish p53 when the predictions were compared across distant species (FIG. 1A). The putative MREs of miR-125b were also found in the 3′ UTRs of p53 in. other vertebrates (FIG. 9A). This suggests that miR-125b is likely to be an important regulator of p53.

We validated the binding of miR-125b to the 3′ UTR of human and zebrafish p53 using a luciferase reporter assay (FIG. 1A, B). Ectopic expression of miR-125b by transfection of miR-125b duplex into HEK-293T cells suppresses by ˜60% (P<0.01) the activity of a Renilla luciferase construct containing the miR-125b MREs of human or zebrafish p53 at its 39 end (FIG. 1B). Similarly, the activity of a luciferase construct containing the entire 3′ UTR of human or zebrafish p53 was suppressed ˜40%-50% (P<0.01) by ectopic miR-125b (FIG. 1B). Suppression of luciferase activity was abolished when the miR-125b-MREs were deleted from the p53 3′ UTR, and when a 3-base mismatch mutation was introduced into the seed region (FIG. 1B). These data indicate that the predicted MREs are critical for the direct and specific binding of miR-125b to the p53 mRNA.

To confirm the binding of miR-125b to human and zebrafish p53 in vitro, we ectopically expressed the full length human/zebrafish p53 cDNA in p53-null H1299 cells. Consistent with the luciferase reporter assays, over-expression of miR-125b in H1299 cells significantly repressed both human and zebrafish wild-type p53 protein (30%-40%, P<0.05) but not when the MREs were deleted from the 39 UTRs of p53 mRNAs (FIG. 1C-E).

Moreover, we showed that the negative regulation of p53 by miR-125b is physiologically relevant to cell function and embryonic development. Ectopic expression of miR-125b is able to repress p53 protein modestly but significantly in human fetal lung fibroblasts and human neuroblastoma SH-SY5Y cells. An increase of miR-125b also represses apoptosis, whereas knockdown of miR-125b increases the level of p53 protein and apoptosis in both in human cells and during zebrafish embryogenesis. In particular, zebrafish embryos displayed a specific neural cell death phenotype upon miR-125b knockdown. Therefore, miR-125b may well be a conserved survival factor that directly or indirectly keeps the level of p53 low during development to support normal tissue growth.

In addition, we validated the specificity and the efficacy of the miR-125b duplex (125b-DP) and the miR-125b antisense oligonucleotide (125b-AS) that were used in our assays. We transfected into HEK-293T cells a luciferase reporter containing a perfect complementary MRE of miR-125b to provide a site with high binding affinity for the miRNA. Indeed, co-transfection of the cells with 125b-DP suppresses the luciferase activity by; 95%, while no suppression was observed with any of the control miRNA duplexes (negative control duplex, NC-DP1/2/3) (FIG. 9B): The luciferase activity suppression by 125b-DP was abrogated when 125b-AS was co-transfected; this effect was not observed with the same concentration of a negative control miRNA antisense oligonucleotide (FIG. 9B). Therefore, 125b-DP and 125b-AS are effective and specific for modulating the level of miR-125b. The transfection efficiency and the effect of these oligonucleotides on miR-125b levels were also verified by quantitative RT-PCR (qRT-PCR) in each cell type. For example, in H1299 cells, we found that the transfection of 125b-DP increased the level of miR-125b by ˜25-fold in comparison with the endogenous miR-125b level (FIG. 9C).

miR-125b Represses Endogenous p53 and p53-Induced Apoptosis in Human Neuroblastoma Cells

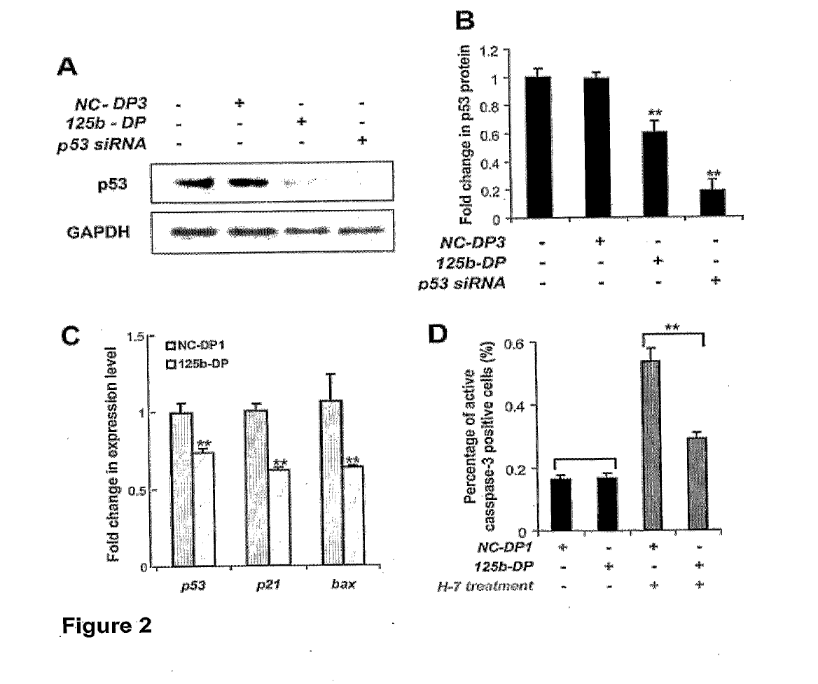

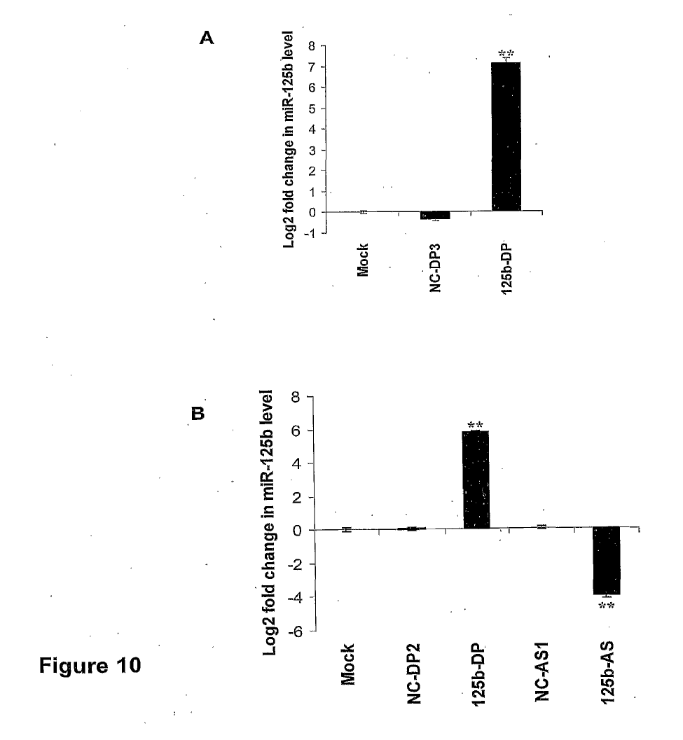

To investigate the regulation of endogenous human p53 by miR-125b, we used the neuroblastoma cell line SHSY5Y, which is known to express wild-type p53 (Vogan et al. 1993). The endogenous level of miR-125b in undifferentiated SH-SY5Y is relatively low, and transfection of miR-125b duplex brought miR-125b level up by ˜27-fold (FIG. 10A). Ectopic expression of miR-125b reduced the level of p53 protein in SH-SY5Y cells by; 40% (P<0.01) (FIG. 2A,B). The level of p53 mRNA was also reduced by 125b-DP transfection although the fold change was smaller than that of p53 protein (FIG. 2C). The expression of p21 and bax, the two main targets of p53, also dropped significantly after 125b-DP transfection (FIG. 2C).

Induction of p53 often leads to apoptosis (Almog and Rotter 1997). However, in neuroblastoma cells, p53 protein is mainly localized to the cytoplasm, so the endogenous activity of nuclear p53 is usually insufficient to modulate apoptosis (Moll et al. 1996). Thus, we predicted that ectopic expression of miR-125b in SH-SY5Y cells will only suppress apoptosis when the p53 pathway is fully activated by an exposure to the drug 1-(5-isoquinolinyl sulfonyl)-2-methyl piperazine (H-7). Exposure to H-7 leads to an increased import of p53 into the nucleus, where p53 becomes active and induces apoptosis (Ronca et al. 1997). Indeed, ectopic expression of miR-125b significantly suppressed H-7-induced apoptosis, but did not affect apoptosis in the untreated SH-SY5Y cells, as quantified by the staining of active-caspase-3 (FIG. 2D).

miR-125b Represses Endogenous p53 and Apoptosis in Primary Human Lung Fibroblasts

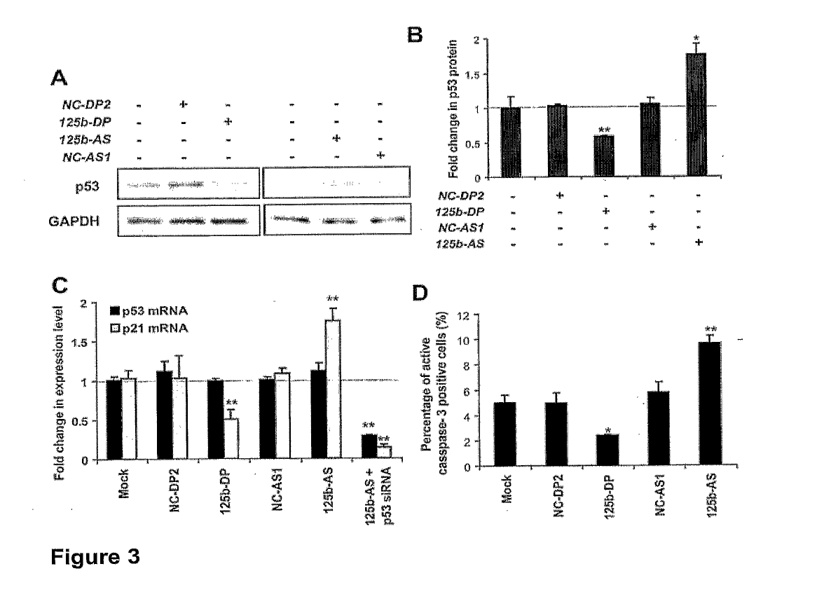

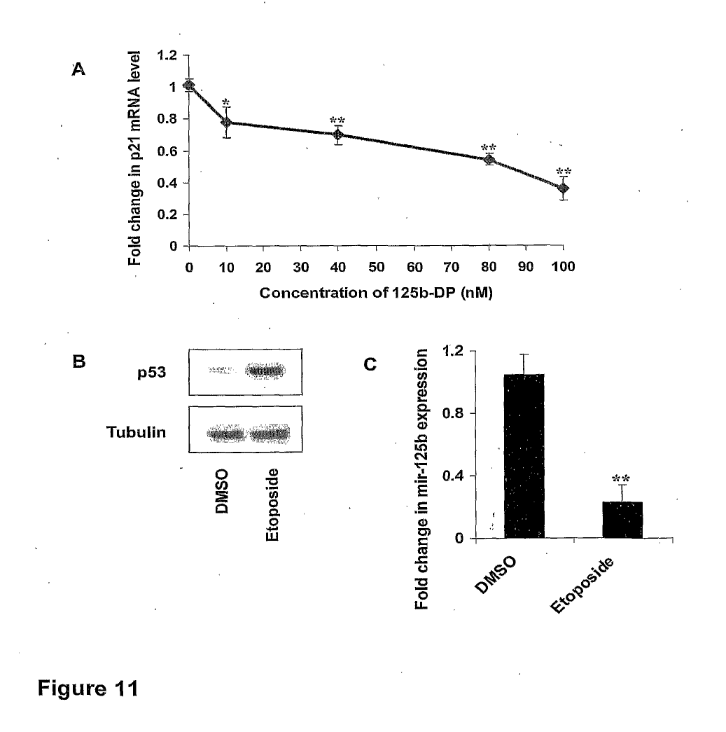

To further demonstrate the repression of human p53 by miR-125b in a physiological context, we examined this regulation in primary human lung fibroblasts that were cultured from normal fetal lungs to homogeneity. The level of miR-125b expression in human lung fibroblasts is relatively high. We were able to knock down the endogenous miR-125b by ˜24-fold with 125b-AS or over-express miR-125b by ˜26-fold with 125b-DP (FIG. 10B). Consistently, over-expression of miR-125b repressed p53 protein levels, while knockdown of miR-125b elevated p53 levels significantly (FIG. 3A,B). The expression of p21 mRNA, a main target of p53, in human lung fibroblasts was also modulated by miR-125b in the same fashion as p53 protein (FIG. 3C). Here, the effect of miR-125b on p21 mRNA level was solely dependent on p53 expression since knockdown of p53 by a siRNA was able to rescue the increase in p21 expression caused by the 125b-AS (FIG. 3C). In addition, 125b-DP represses p21 expression in a dose-dependent manner, with significant suppression still observable at a concentration as low as 10 nM of 125b-DP (FIG. 11A). The level of p53 mRNA in human lung fibroblasts, however, was not affected by the changes in miR-125b expression (FIG. 3C). This suggests that miR-125b inhibits the translation of p53 but does not modulate the stability of p53 mRNA in these cells. In addition, miR-125b knockdown led to a substantial increase in apoptotic cells, as quantified by active-caspase-3 staining, while miR-125b over-expression had the opposed effect (FIG. 3D). These data demonstrate that miR-125b expression is both necessary and sufficient for maintaining the physiological levels and the activity of p53 in human lung fibroblasts.

A reduction in miR-125b level is also important for the in vivo p53-dependent stress response. We demonstrated that miR-125b was down-regulated in zebrafish embryos just 8 h after γ-irradiation or camptothecin treatment, while p53 protein level was elevated dramatically. Furthermore, the DNA damage stress-induced p53 and apoptosis in zebrafish embryos was repressed by miR-125b ectopic expression. Similarly, we also observed a down-regulation in miR-125b in human neuroblastoma SH-SY5Y cells after a 24-h treatment with etoposide, a topoisomerase inhibitor known to induce DNA damage and activate p53 (FIG. 11B, C). Our data suggest that the down-regulation of miR-125b in response to the stress of DNA damage is conserved in both zebrafish and humans. It would be interesting to further characterize the mechanism of miR-125b down-regulation in response to DNA damage.

### Specificity Versus Off-Target Effects

We demonstrated that miR-125b acts as a direct negative regulator of p53 in human and zebrafish. The induction of p53 and the increase in apoptosis is an expected phenotype of miR-125b knockdown. However, the dramatic effects of the miR-125b morpholinos in zebrafish raised concerns of a possible off-target effect.

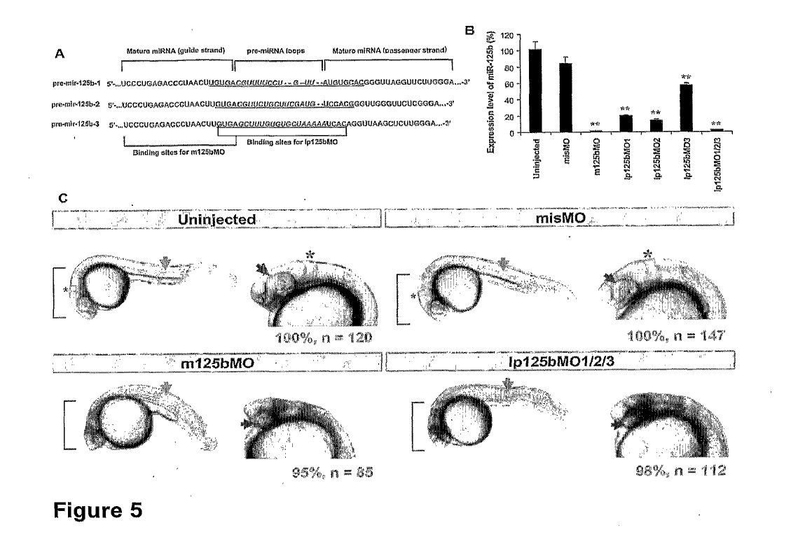

In order to address the specificity of miR-125b morpholinos, we followed two approaches. First, two different sets of morpholinos were used to knock down miR-125b: the m125bMO (targets the mature miR-125b) and the three lp125bMOs (target the loop region of the three pre-mir-125b isoforms). The sequence of m125bMO overlaps with the lp125bMOs by only 3-4 nucleotides (nt) (FIG. 5A). Thus, the probability of all of these morpholinos having the same off-target effect is very low (2%-4%). Second, and importantly, we were able to rescue miR-125b morphants specifically with synthetic miR-125b duplex, suggesting that the morphant phenotype is an on target effect of the morpholinos'.

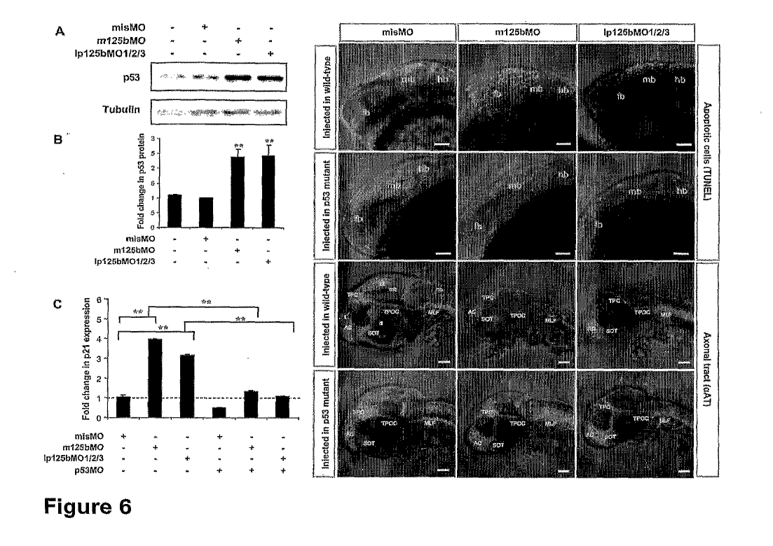

In the first approach, our data showed that knockdown of miR-125b by either m125bMO or lp125bMOs resulted in the same phenotype: up-regulation of p53 protein and increase in neural cell death at 24 hpf (FIGS. 5C, 6A-D). In fact, the severity of the phenotype was dependent on the dose and the efficacy of the morpholinos. For m125bMO or for the combination of lp125bMO1/2/3, injection of 0.25 pmol of morpholino had no effect; 0.5 pmol of morpholino induced a mild neural cell death in ˜80% of injected embryos; and 0.75 pmol of morpholino produced severe neural cell death in ˜95% of the embryos. m125bMO reduced the level of mature miR-125b to almost zero (FIG. 5B), and the apoptotic phenotype was the most severe in embryos injected with this morpholino. Injection of all three lp125bMOs resulted in a comparable effect to that of m125bMO because this coinjection would inhibit the processing of all pre-mir-125b isoforms. Injection of each lp125bMO individually resulted in an incomplete knockdown of miR-125b (FIG. 5B) and led to a mild neural cell death. In particular, the level of mature miR-125b was reduced more by lp125bMO1 and lp125bMO2 than by lp125bMO3, probably due to the lower expression of pre-mir-125b-3 in the embryos. As a consequence, injection of lp125bMO1 or lp125bMO2 resulted in more cell death than that of lp125bMO3. In addition, to test whether sequence specificity was important for knockdown of miR-125b, we designed a control morpholino that had the same length and GC content as the m125bMO but contained five mismatches. This mismatched morpholino (misMO) was not able to knock down miR-125b (FIG. 5B). Moreover, embryos injected with misMO exhibited no difference in morphology from those of uninjected controls (FIG. 5C).

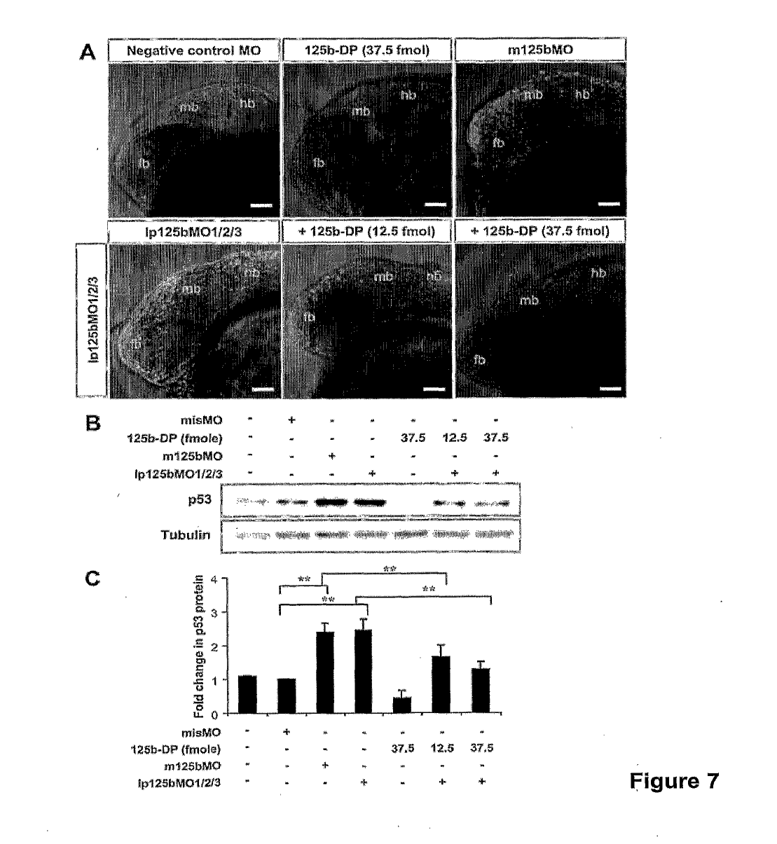

The specificity of knockdown was further demonstrated by the second approach: Over-expression of miR-125b by injection of synthetic miR-125b duplex rescued the effect of lp125bMO1/2/3 in a dose-dependent manner. Specifically, injection of 37.5 fmol of the miR-125b duplex reduced the level of p53 protein and the number of apoptotic cells significantly (FIG. 7A-C). With this dose, the percentage of embryos with neural cell death dropped from 98% in the lp125bMO1/2/3 morphant population, to 6% in the rescued embryos in which lp125bMO1/2/3 was coinjected with the miR-125b duplex (Table 1). Because lp125bMO1/2/3 can only bind to the loop regions of mir-125b precursors and block processing of endogenous miRNA precursors, it cannot interact with the synthetic miR-125b duplex. Moreover, lp125bMO1/2/3 was injected at a 20- to 30-fold (750:37.5 to 750:12.5) higher dose than the miR-125b duplex; hence, the rescue could not be due to a titration of the morpholino. Instead, this result implies that the mature miR-125b processed from the injected synthetic duplex was able to replenish endogenous miR-125b, and thus repress p53 and down-regulate apoptosis in the embryos.

TABLE 1
Percentage of embryos with neural cell death

| | | | | | | |
|---|---|---|---|---|---|---|
| Negative control MO | + | − | − | − | − | − |
| 125b-DP (fmol) | − | 37.5 | − | − | 12.5 | 37.5 |
| m125bMO | − | − | + | − | − | − |
| lp125bMO1/2/3 | − | − | + | − | − | − |
| Total survived embryos | 91 | 95 | 85 | 112 | 90 | 121 |
| Embryos with neural | 0% | 0% | 95% | 98% | 54% | 6% |
| cell death | | | | | | |

Neural cell death was observed in 24-hpf live embryos as the accumulation of dark cells in the brain (example shown in FIG. 5C). The embryos were counted in a double-blind manner.

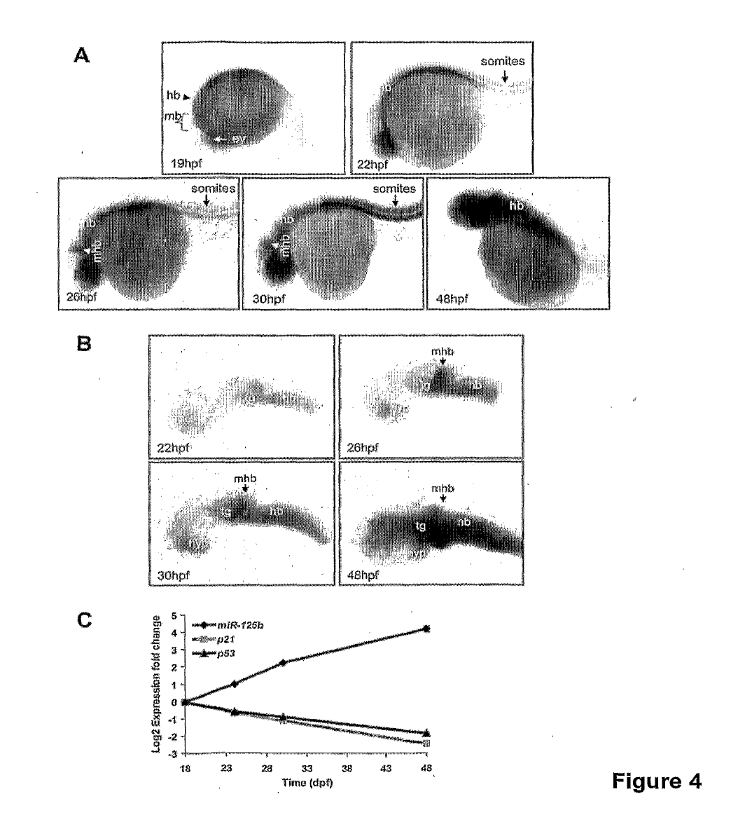

These two lines of evidence strongly suggest that the miR-125b morpholinos used in our experiments were on target. Additionally, the phenotype of the miR-125b morphants is also explained by the temporal and spatial expression pattern of miR-125b. Defects in the miR-125b morphants appear precisely in the regions where miR-125b is normally strongly expressed; e.g., the brain, the eyes, and the somites (cf. FIGS. 5C and 4A). The onset of increased apoptosis in the morphants is also correlated to the stage (18 hpf) when miR-125b begins to express in the embryos (cf. Supplemental FIG. 5 and FIG. 4C).

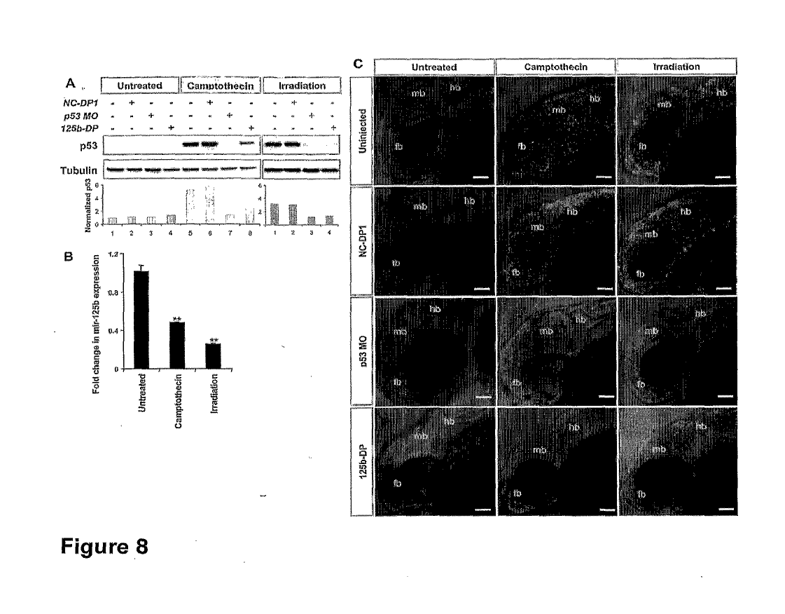

Moreover, the expression of miR-125b was up-regulated during development, while the activity of p53 was decreasing (FIG. 4C). In contrast, miR-125b was down-regulated in response to DNA damage, while p53 was up-regulated under the same condition (FIG. 8). The inverse correlation between miR-125b expression and p53 strengthens our conclusion that miR-125b is a physiological regulator of p53.

Conservation of miR-125b Targets in the p53 Network

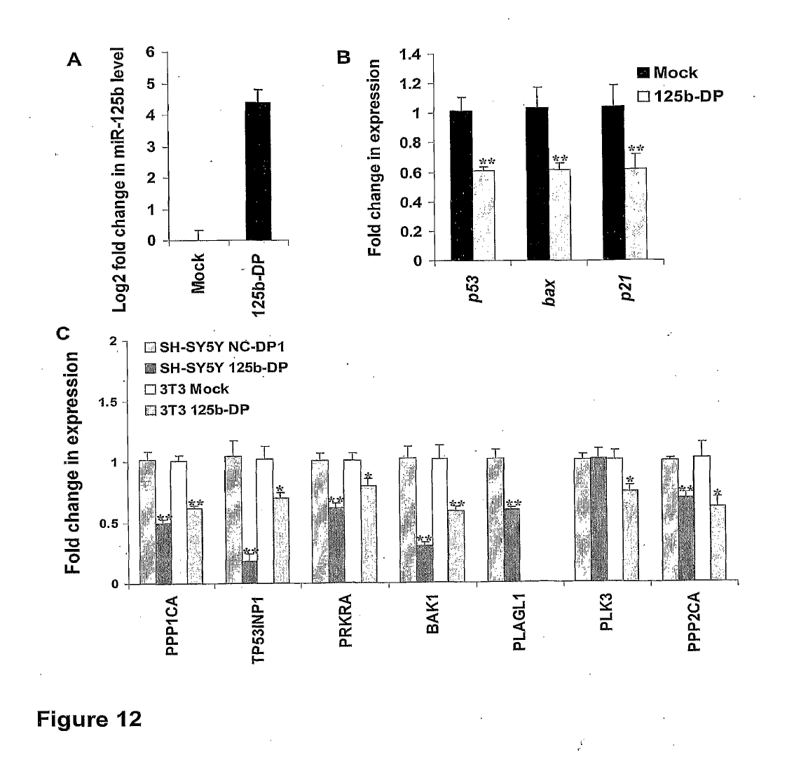

Besides p53, miR-125b may also target other components of the p53 network. The mRNAs of these genes all contain putative binding sites for miR-125b in their 3′ UTRs. We compared the putative binding sites of the seven targets across a number of vertebrates and found that each site is broadly (although not strictly) conserved among vertebrates (Table 2). Each species has a binding site for miR-125b in the sequence of at least one of the seven targets. Supporting these findings, we demonstrated that these seven genes in the p53 network were indeed downregulated by miR-125b ectopic expression in the human neuroblastoma SH-SY5Y cells and/or in mouse fibroblast Swiss-3T3 cells (FIG. 12C). Hence, these genes are likely to be targets of endogenous miR-125b in both human and mouse. Furthermore, miR-125b ectopic expression also down-regulated p53 mRNA as well as the p53 targets, p21 and bax mRNAs in mouse fibroblast Swiss-3T3 cells. Since the binding site for miR-125b in the 3′ UTR of p53 mRNA is not conserved in mouse, the down-regulation of p53 expression by miR-125b may be mediated indirectly by the down-regulation of the genes upstream of p53 in mouse Swiss-3T3 cells. Although further experiments are required to validate the direct targets of miR-125b in the p53 network, our analysis strongly suggests that the p53 network, as a whole, is a broadly conserved target of miR-125b regulation in vertebrates.

TABLE 2
Putative miR-125b targets in the p53 pathway

| Targets | TP53 | PRKRA | PLK3 | TP53INP1 | BAK1 | PLAGL1 | PPP1CA | PPP2CA |
|---|---|---|---|---|---|---|---|---|
| Human | + | + | + | + | + | + | + | + |
| Monkey | + | + | + | + | − | + | + | + |
| Mouse | − | + | + | + | + | + | + | + |
| Guinea Pig | − | − | − | + | + | − | + | + |
| Rabbit | + | + | − | + | + | + | + | + |
| Cat | − | − | + | + | + | − | + | − |
| Horse | + | + | + | + | + | + | + | + |
| Opossum | − | − | − | + | − | + | − | + |
| Lizard | − | − | − | + | + | − | − | − |
| Chicken | + | − | − | − | − | − | − | + |
| Frog | + | − | − | − | − | − | − | + |
| Zebrafish | + | − | − | − | − | − | − | − |

Most miRNAs and their mRNA-binding motifs (the seed regions) are strictly conserved across species, but their targets are less well-conserved. The loss/gain during evolution of an individual mRNA target may make very little impact on the function of a miRNA with multiple targets. We found that miR-125b targets multiple genes in the p53 network, where the redundancy of these targets allows for their relatively neutral loss/gain across various species. While not every target is strictly conserved, the overall regulation of p53 activity by miR-125b may still be conserved during evolution via one or another component of the p53 network.

Other Targets and Other Functions of miR-125b

Genes in the p53 network are most probably not the only targets of miR-125b. We observed several defects in zebrafish embryos after ectopic expression of miR-125b, including a delay in growth, rounding of the body, thickening of yolk extension, and loss of brain ventricles and of the midbrain-hindbrain boundary (data not shown). These phenotypic effects are not observed in p53-deficient zebrafish. In addition, we also found that ectopic expression of miR-125b promotes neuronal differentiation in human neural progenitors and neuroblastoma cells. This phenotype was not recapitulated by knockdown of p53. Additional targets that mediate the function of miR-125b in zebrafish and in human neural cells remain to be identified.

Spatio-Temporal Expression of miR-125b During Zebrafish Embryogenesis

To examine if miR-125b expression is inversely correlated to p53 expression spatio-temporally during development, we analyzed miR-125b expression at different stages of zebrafish embryogenesis. Expression of miR-125b was first detected at 19 h post-fertilization (hpf) by whole mount in situ hybridization (FIG. 4A). miR-125b was present in the whole embryo with enrichment in the brain, the eyes, and the somites at different stages (FIG. 4A,B). The expression pattern in the brain is consistent with previously published data (Wienholds et al. 2005). However, no enriched expression was detected in the spinal cord. Instead, we found a pronounced miR-125b expression in the somites between 22 and 30 hpf (FIG. 4A). By 22 hpf, miR-125b expression was enriched in the eyes, the somites, the telencephalon, and the midbrain with stronger expression in the tegmentum and hindbrain (FIG. 4A,B). Between 26 and 30 hpf, miR-125b was strongly expressed in the hypothalamus, the tegmentum, the midbrain-hindbrain boundary, and the hindbrain (FIG. 4B). miR-125b expression continues to increase in the brain such that the optic tectum became the only region with weak miR-125b expression by 48 hpf (FIG. 4A,B).

Analysis of miR-125b expression in whole embryo lysate by qRT-PCR showed that the expression initiated at 18 hpf and increased exponentially from 18 to 48 hpf (FIG. 4C). Interestingly, p53 and p21 expression was inversely correlated with miR-125b up-regulation over time (FIG. 4C). We also compared the spatio-temporal expression pattern of miR-125b (our in situ hybridization analysis) with the expression pattern of p53 mRNA (Yamaguchi et al. 2008). p53 and miR-125b were observed to be co-expressed in the brain and the eyes at; 24 hpf. In the brain, miR-125b expression increases steadily from 24 to 48 hpf, while p53 expression decreases gradually during that same period. In the somites, miR-125b expression is enriched from 22 to 30 hpf, while p53 expression is not observed. Western blots also showed that p53 protein can be detected at 18 hpf and decreases to undetectable levels by 48 hpf (data not shown). The inverse correlation between miR-125b and p53 expression/activity supports our hypothesis that p53 is down-regulated by miR-125b during zebrafish embryogenesis.

Loss of miR-125b Leads to Severe Defects in Zebrafish Embryos

To probe for the function of miR-125b in zebrafish, we synthesized four different morpholinos against miR-125b (FIG. 5A): one (m125bMO) targeting the mature guide strand, and three (lp125bMOs) targeting the precursors. In zebrafish, mature miR-125b is derived from three different precursor isoforms with sequence differences in the loop region (FIG. 5A); lp125bMOs were designed to bind to each of these loops. Binding of the morpholinos to the loop regions of miRNA precursors is considered able to block processing of the miRNAs, hence down-regulating the mature miRNA level. Near-complete knockdown of mature miR-125b was observed with m125bMO and also with a combination of the three lp125bMOs (FIG. 5B). Individual lp125bMOs also suppressed the expression of miR-125b, albeit incompletely (FIG. 5B). As a control, injection of a morpholino (misMO) with five mismatches different from m125bMO did not cause in any significant change in miR-125b expression (FIG. 5B).

Severe developmental defects were observed in the miR-125b morphants, where the most apparent phenotype was the accumulation of dead cells in the brain (FIG. 5C). This phenotype was observed by 24 hpf in almost all embryos microinjected with m125bMO or with lp125bMO1/2/3 (FIG. 5C; Table 1). Other morphological defects upon miR-125b knockdown include smaller eyes, a missing midbrain-hindbrain boundary, and deformities in the somites. These data are consistent with the expression pattern of miR-125b and demonstrate its importance in zebrafish development.

Loss of miR-125b Increases p53 and p53-Dependent Apoptosis in Zebrafish

As a result of miR-125b knockdown by injection of m125bMO or lp125bMO1/2/3 into one-cell-stage embryos, the endogenous level of p53 protein was elevated in zebrafish embryos at 24 hpf (FIG. 6 A, B). p21 was also up regulated in both types of morphants (FIG. 6 C). When the morphants were coinjected with a morpholino blocking translation of p53, p21 expression was restored to wild-type levels, indicating that the up-regulation of p21 by miR-125b required p53 (FIG. 6 C).

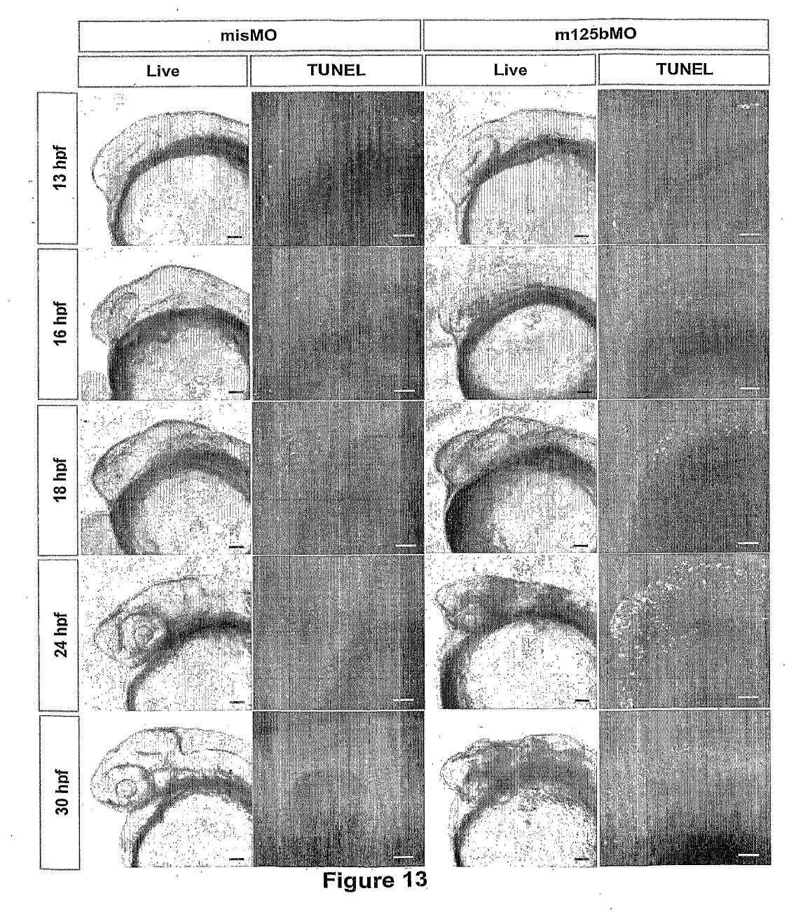

At 24 hpf, an increase in terminal dUTP nick end labeling (TUNEL)-positive apoptotic cells was observed in the midbrain and hindbrain domains of both m125bMO- and lp125bMO1/2/3-injected embryos (FIG. 6 D). Enhanced apoptosis was observed in the m125bMO morphants only from 18 hpf, consistent with the stage when miR-125b expression was first detected (FIG. 13). Apoptosis reached a peak at 24 hpf, when the brain defects were the most severe (FIG. 13). Apoptosis decreased gradually by 30 hpf (FIG. 13), but the hatched larvae were still defective, with distorted heads and abnormal behaviors.

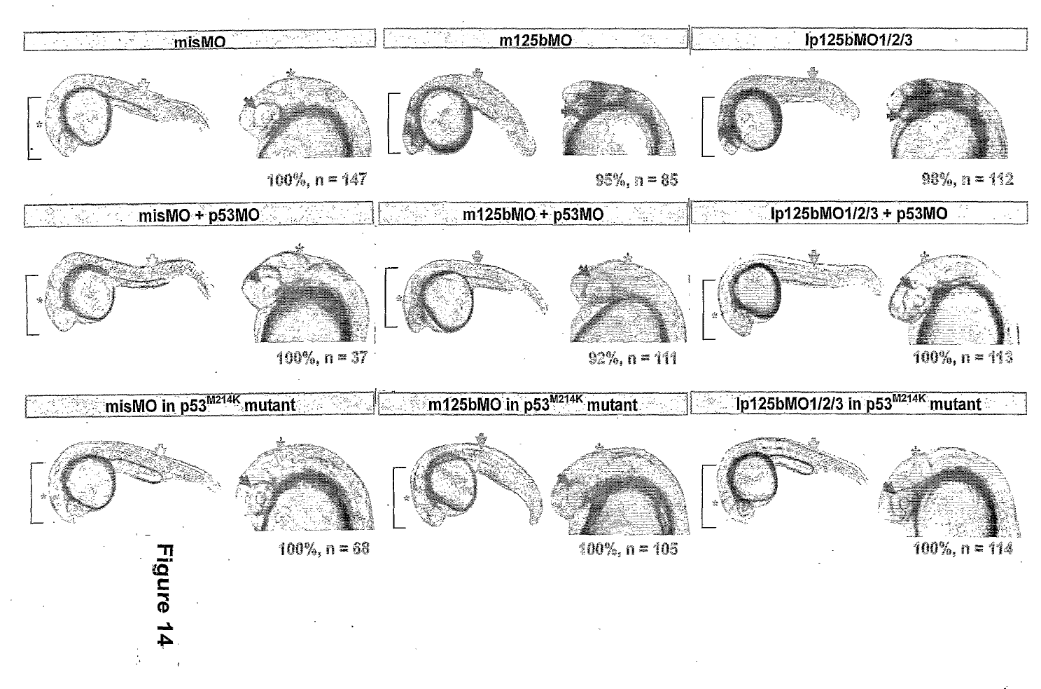

We then asked whether the cell death phenotype in miR-125b morphants was caused by the elevation in p53 protein. To ablate p53 function, we used the zebrafish p53M214K mutant, which is defective in p53 activity but still undergoes normal embryogenesis (Berghmans et al. 2005). Remarkably, knockdown of miR-125b, whether by m125bMO or by lp125bMO1/2/3, had no observable effects on brain apoptosis (FIG. 6 D). Similar effect was observed with the co-injection of p53 MO and miR-125b MOs (FIG. 14). Defects in the midbrain-hindbrain boundary and the somites of miR-125b morphants were also rescued in p53M214K mutants or by co-injection of p53 MO (FIG. 14). Additionally, miR-125b morphants exhibited severe defects in axonal path finding, as observed by anti-acetylated tubulin immunostaining (FIG. 6D). Most major primary axonal tracts were markedly reduced in the miR-125b morphants, but they were rescued substantially by the loss of p53-mediated apoptosis in the p53M214K mutant (FIG. 6 D). Taken together, these data demonstrate that the excessive p53 activity in miR-125b morphants is responsible for the abnormal increase in apoptosis and most of the observed morphological defects. Therefore, the p53 pathway is likely to be the major target that mediates the function of miR-125b during the early development of zebrafish.

Synthetic miR-125b Duplex Rescues Apoptosis in miR-125b Morphants by Restoring the Normal Level of p53

In order to address the specificity of miR-125b morpholinos, we attempted to resale the morphant phenotype with co-injection of synthetic miR-125b duplex into lp125bMO1/2/3 morphants. Indeed, the number of apoptotic cells in the morphants was reduced significantly by miR-125b duplex in a dose-dependent manner (FIG. 7A). The efficiency of rescue was quantified by the number of embryos with visible dead cells in the live brain at 24 hpf. Both m125bMO and lp125bMO1/2/3 injection caused more than 90% of embryos to show neural cell death, whereas co-injection of the miR-125b duplex reduced this number to only 6% (Table 1). This rescue of apoptosis corresponds to the reduction in p53 protein by miR-125b duplex (FIG. 7 B, C) Importantly, since this duplex does not bind to the lp125bMOs, rescue of the lp125bMO1/2/3 morphants demonstrates that synthetic miR-125b duplex produced mature miR-125b that replenished the endogenous miR-125b to repress p53 and down-regulate apoptosis.

Stress-Induced p53 and Apoptosis are Repressed by Ectopic miR-125b

To further elucidate the role of miR-125b in zebrafish development, we examined the ability of miR-125b to suppress p53 during the stress response in zebrafish embryos. p53 can be induced quickly by agents that cause DNA damage, leading to cell cycle arrest and apoptosis (Kuerbitz et al. 1992; Langheinrich et al. 2002). To induce DNA damage, we irradiated zebrafish embryos with 40 Gy of γ-rays or treated them with 500 nM camptothecin for 8 h. As expected, the p53 protein increases dramatically after both treatments in wild-type embryos (FIG. 8A). Interestingly, both treatments resulted in a significant drop in miR-125b expression (FIG. 8B), suggesting that the down-regulation of miR-125b allows a smooth up-regulation of p53 in this stress response pathway.

To test whether an ectopic expression of miR-125b can reduce the extent of the DNA damage stress response, we exposed miR-125b-duplex-injected embryos to γ-irradiation or camptothecin treatment. As anticipated, the level of p53 protein in the treated embryos was reduced significantly by miR-125b duplex (FIG. 8A). Staining of apoptotic cells in the embryonic brain further demonstrated that the severe apoptosis induced by γ-irradiation or camptothecin was rescued significantly by the injection of miR-125b duplex (FIG. 8C). In fact, the rescue effects of miR-125b duplex during the DNA damage response were nearly as great as the effects of p53 knockdown via a morpholino (FIG. 8C). A negative control duplex had no effect (FIG. 8C).

The Implication of miR-125b in Tumorigenesis

Our study supports the notion that miR-125b acts as an oncogene by negatively regulating p53 and suppressing p53-dependent apoptosis. miR-125b-p53 interaction represents a new mechanism for regulation of apoptosis useful in certain types of cancer.

### Materials and Methods

### Cloning and Mutagenesis of the Luciferase Reporters and the p53 Expression Constructs

The MREs or the whole 3′ UTR of p53 were cloned into the psiCHECK-2 vector (Promega), between the XhoI and NotI sites, immediately 3′ downstream from the Renilla luciferase gene. The top (sense) and bottom (antisense) strands of each MRE were designed to contain XhoI and NotI sites, respectively (Table 3). They were synthesized, annealed, and ligated into the psiCheck-2 vector. The 3′ UTR of human p53 was amplified from the total cDNA of SH-SY5Y cells by a nested PCR and inserted into TOPO PCR2.1 (Invitrogen). Subsequently, the UTR was released from the TOPO vector by XhoI and NotI and ligated into the psiCheck2 vector. The 3′ UTR of zebrafish p53 was amplified from total cDNA of 18-hpf zebrafish embryos, digested with XhoI/NotI, and ligated directly into the psiCheck2 vector. The full-length cDNA encoding human and zebrafish p53 was cloned into a pcDNA3.1⁺ vector, in between the EcoRI and XhoI sites, downstream from the CMV promoter. The human p53 cDNA was PCR-amplified from the total cDNA of SH-SY5Y cells. The zebrafish p53 cDNA was PCR-amplified from the total cDNA of 18hpf zebrafish embryos, using fp53Fe/fp53Rx primers.

Deletion or mutation of the miR-125b MRE in the p53 construct was performed using the QuickChange site-directed mutagenesis kit (Promega) according to the manufacturer's instructions. The sequences of all primers are provided in Table 3.

TABLE 3
Primer sequences

| Name | Type | Sequence |
|---|---|---|
| Luc-hP53-mreF | MRE top strand | 5′-TCGAGAAGACTTGTTTTATGCTCAGGGTGC-3′ |
| Luc-hP53-mreR | MRE bottom strand | 5′-GGCCGCACCCTGAGCATAAAACAAGTCTTC-3′ |
| Luc-fP53-mreF | MRE top strand | 5′-TCGAGTGAAATGTCAAATACTCAGGGCAGC-3′ |
| Luc-fP53-mreR | MRE bottom strand | 5′-GGCCGCTGCCCTGAGTATTTGACATTTCAC-3′ |
| hP53-UTR-F1 | PCR forward primer | 5′-AAGTCCAAAAAGGGTCAGTCTACCTCCC-3′ |
| hP53-R1 | PCR reverse primer | 5′-GCTCACAATTGTAATCCCAGCACTCTGG-3′ |
| hP53-UTR-Fx | PCR forward primer | 5′-CCGCTCGAGACCCAGGACTTCCATTTGCTTTGTC-3′ |
| hP53-UTR-Rn | PCR reverse primer | 5′-TTCCTTTTGCGGCCGCGATCGCCTGAGCCCAGGA |
| | | GTTT-3′ |
| fP53-UTR-Fx | PCR forward primer | 5′-CCGCTCGAGATG CTA AGA GAG AAA GAA ACT GG3′ |
| fP53-UTR-Rn | PCR reverse primer | 5′-TTCCTTTTGCGGCCGCGCAAATGCGTGTAAACAGTA |
| | | ATAAG-3′ |
| hp53-F1 | PCR forward primer | 5′-GTCATGGCGACTGTCCAGCTTTGTG-3′ |
| hp53-Fe | PCR forward primer | 5′-CCGGAATTCGTTCGGGCTGGGAGCGTGCTTT-3′ |
| hp53-Rx | PCR reverse primer | 5′-CCGCTCGAGTCTGGGAGGCTGAGACAGGTGGAT-3′ |
| fp53-Fe | PCR forward primer | 5′-CCGGAATTCGTTTAGTGGAGAGGAGGTC-3′ |
| fp53-Rx | PCR reverse primer | 5′-CCGCTCGAGAGTTTTATCAAGATTTCCAGGAGG-3′ |
| hp53XhoMutF | Mutagenesis primer | 5′-CAGCCTTTGCCTCCCCGGGtGgTgCAGTCCTGCCTC |
| | | AGCC-3′ |
| hp53XhoMutR | Mutagenesis primer | 5′-GGCTGAGGCAGGACTGCACCACCCGGGGAGGCAAAG |
| | | GCTG-3′ |
| hP53-3misF | Mutagenesis primer | 5′-GGATCCACCAAGACTTGTTTTATGCTgcGGcTC |
| | | AATTTCTTTTTTC-3′ |
| hP53-3misR | Mutagenesis primer | 5′-GAAAAAAGAAATTGAGCCGCAGCATAAAACAAGTC |
| | | TTGGTGGATCC-3′ |
| hP53delF | Mutagenesis primer | 5′-GTATATGATGATCTGGATCCACCAAGTCAATTTCTTT |
| | | TTTC3′ |
| hP53delR | Mutagenesis primer | 5′-GAAAAAAGAAATTGACTTGGTGGATCCAGA |
| | | TCATCATATAC-3′ |
| fP53-3misF | Mutagenesis primer | 5′-GCAAATGAAATGTCAAATACTgAcGcCATGTACA |
| | | AGTCCCTCC-3′ |
| fP53-3misR | Mutagenesis primer | 5′-GGAGGGACTTGTACATGGCGTCAGTATTTGACATTT |
| | | CATTTGC-3′ |
| fP53-delF | Mutagenesis primer | 5′GATTGGATGTCTAAATATGAGCAAATGTACAAATG |
| | | TACAAGTCCCTCCTGGAAATCTTGATAAAAC-3′ |
| fP53-delR | Mutagenesis primer | 5′GTTTTATCAAGATTTCCAGGAGGGACTTGTACATTTG |
| | | TACATTTGCTCATATTTAGACATCCAATCC-3′ |
| fActinF SYBR | forward primer | 5′-CCAGACATCAGGGAGTGAT-3′ |
| fActinR SYBR | reverse primer | 5′-TCTCTGTTGGCTTTGGGATT-3′ |
| fp21F SYBR | forward primer | 5′-CGGAATAAACGGTGTCGTCT-3′ |
| fp21R SYBR | reverse primer | 5′-CGCAAACAGACCAACATCAC-3′ |
| hp21F SYBR | forward primer | 5′-TTTCTCTCGGCTCCCCATGT-3′ |
| hp21R SYBR | reverse primer | 5′-GCTGTATATTCAGCATTGTGGG-3′ |
| hbaxF SYBR | forward primer | 5′-CCGATTCATCTACCCTGCTG-3′ |
| hbaxR SYBR | reverse primer | 5′-CAATTCCAGAGGCAGTGGAG-3′ |

Grey text indicates restriction enzyme sites; grey underlined text indicates the overhang sequences for binding of restriction enzymes; lower case text indicates mismatched mutations.

### Luciferase Reporter Assay

miRNA duplexes including negative control duplex 1 and 2 (NCDP1/2, negative control PremiR #1 and #2), negative control duplex 3 (NC-DP3, miR-7 PremiR), and miR-125b duplex (125b-DP, miR-125b PremiR); miRNA antisense oligonucleotide including negative control antisense 1 (NC-AS1, negative control AntimiR 1) and miR-125b antisense (125b-AS, miR-125b AntimiR) were purchased from Ambion and dissolved in water. miR-7 duplex was used as a negative control in our experiments because no seed match of miR-7 of can be found in the 3′ UTR of human and zebrafish p53 mRNAs. Ten nanograms of each psiCHECK-2 construct were co-transfected with 10 nM miRNA duplexes or 100 nM miRNA antisenses into HEK-293T cells in a 96-well plate using lipofectamin-2000 (Invitrogen). After 48 h, the cell extract was obtained; firefly and Renilla luciferase activities were measured with the Dual-Luciferase reporter system (Promega) according to the manufacturer's instructions.

### Cell Culture, Transfection, and Drug Treatments

Human HEK-293T cells, human neuroblastoma SH-SY5Y cells, p53-null human lung carcinoma H1299 cells, mouse Swiss-3T3 cells, and human lung fibroblast cells were maintained in DMEM or RPMI media, supplemented with 10% fetal bovine serum and 1% penicillin-streptomycin (Invitrogen). H1299 cells, SH-SY5Y cells, Swiss-3T3 cells, and human lung fibroblast cells were transfected in suspension with 4×10⁵ cells per well in six-well plates using lipofectamin-2000 (Invitrogen). Plasmids (human/zebrafish wildtype or mutant p53 constructs) were transfected into H1299 cells at a final concentration of 0.5 μg/mL. miRNA duplexes and antisense oligonucleotides were transfected at a final concentration of 80 nM and 100 nM, respectively (unless otherwise stated). p53 siRNA (Dharmacon) was transfected at 60 nM final concentration.

H-7 and etoposide (Sigma) were dissolved in water and dimethyl sulfoxide (DMSO), respectively. SH-SY5Y cells (untransfected or 24 h after transfection with miRNA duplexes) were treated with 10 μM H-7 or 10 μM etoposide for 24 h. Control cells were treated with water or DMSO, respectively.

### Active Caspase-3 Assay

Two days after transfection, SH-SY5Y cells and human lung fibroblast cells were fixed with 4% paraformaldehyde and treated with cold methanol for 10 min at −20° C. After 1-h blocking with 0.3% Triton X-100 and 3% goat serum in phosphate-buffered saline (PBS), the cells were incubated with anti-active-caspase-3 antibody (BD Biosciences) overnight at 4° C. and then incubated with Alexa Fluor 568 goat anti-rabbit secondary antibody (Invitrogen) and Hoechst (Invitrogen). Fluorescent images of the cells were collected and analyzed by the Cellomics high content screening system.

### Whole Mount In Situ Hybridization

Whole mount in situ hybridizations with double-Dig-labeled miR-125b miRCURY LNA probe (Exiqon) on zebrafish embryos were performed essentially as described (Wienholds et al. 2005). Modifications to the protocol include an incubation of the 19-, 22-, and 24-hpf embryos for 30 sec and of the 30-hpf embryos for 1 min with PCR-grade proteinase K (Roche) after fixing. The hybridization mix was prepared by adding 20 pmol of miR-125b doubled-labeled LNA probe to every 1 mL of hybridization solution. The hybridization temperature used was 20° C. below the melting temperature of the miR-125b LNA probe. Optimal signal-to-noise ratio during color development was obtained by washing the embryos with 53 Tris-buffered saline containing 0.1% Tween 20 (TBST buffer) between color reactions. This cycle of washing with 53 TBST followed by color development was repeated thrice. As a control, the same protocol was used for double-Dig-labeled miR-7 miRCURY LNA probe (Exiqon) on zebrafish embryos, and no signal was observed before 48 hpf, consistent with the prior report (Wienholds et al. 2005).

Microinjection in Zebrafish Embryos

Wild-type and p53M214K mutant zebrafish were maintained by standard protocols (Brand et al. 2002). All injections were carried out at the one- to four-cell stage with 2 mL of solution into each embryo. In the knockdown experiments, miR-125b morpholinos were injected at 0.75 pmol per embryo (lp125bMMO1/2/3 indicates the co-injection of three lp125bMOs, 0.25 pmol each); p53 morpholino was co-injected at 1 pmol per embryo; miR-125b duplex was injected at 12.5 fmol or 37.5 fmol per embryo. In the stress response experiments, the embryos were injected with 37.5 fmol per embryo of NC-DP1 or 125b-DP or with 0.5 pmol per embryo p53 MO. Sequences of all morpholinos (GeneTools) are shown in Table 4.

TABLE 4
Morpholino sequences

| Name | Type | Sequence |
|---|---|---|
| m125bMO | Blocking mature mir-125b | 5′-TCACAAGTTAGGGTCTCAGGGA-3′ |
| misMO | Mismatched control | 5′-TCAgAAGaTAGGcTCTCtGGcA-3′ |
| lp125bMO1 | Blocking pre-mir-125b-1 | 5′-GTGCACATAACAGGAAAACGTCACA-3′ |
| lp125bMO2 | Blocking pre-mir-125b-2 | 5′-CGTGGACATCGAAGCAGAACGTCAC-3′ |
| lp125bMO3 | Blocking pre-mir-125b-3 | 5′-GTGATTTTTAGCACACAAAGCTCAC-3′ |
| p53MO | Blocking p53 translation | 5′-GCGCCATTGCTTTGCAAGAATTG-3′ |
| Negative | Standard negative control | 5′-CCTCTTACCTCA GTTACA ATTTATA 3′ |
| control MO | | |

qRT-PCR

RNA was extracted from cells or zebrafish embryos using Trizol reagent (Invitrogen) and subsequently column-purified with RNeasy kits (Qiagen). For qRT-PCR of miR-125b, 100 ng of total RNA was reverse-transcribed and subjected to Taqman miRNA assay (Applied Biosystems). For qRT-PCR of mRNAs, cDNA synthesis was performed with 1 μg of total RNA using the High Capacity cDNA Archive Kit (Applied Biosystems). Subsequently, human p53 and zebrafish 18S expression was analyzed by Taq-Man assay; the expression of all other genes was analyzed by SYBR assay (Applied Biosystems) following the manufacturer's protocol.

### Western Blot Assay

Cells were lysed in RIPA buffer (Pierce). Zebrafish embryos were dechorionated, deyolked, and homogenized in T-PER reagent (Thermo Fisher Scientific) containing protease inhibitor (Roche). Protein was separated by a 10% polyacrylamide gel and transferred to a methanol-activated PVDF membrane (GE Healthcare). The membrane was blocked for 1 h in PB ST containing 7.5% milk and subsequently probed with 0.5 μg/mL anti-p53 antibody (Santa Cruz Biotechnologies) or 1.4 μg/mL Zfp53-9.1 antibody (Lee et al. 2008), anti-Glyceraldehyde-3-phosphate dehydrogenase (GAPDH) antibody (Abcam), or 1 μg/mL anti-a-tubulin antibody (Sigma Aldrich) overnight at 4° C. After 1-h incubation with goat-anti-mouse HRP-conjugated secondary antibody (Santa Cruz Biotechnologies), the protein level was detected with luminol reagent (Santa Cruz Biotechnologies). Intensity of the protein bands was quantified using ImageJ.

### TUNEL Assay

Embryos were dechorionated and fixed in 2% paraformaldehyde (Fluka) overnight at 4° C. They were then dehydrated in methanol (50%, 75%, 95%, 100% series) and incubated with cold acetone for 10 min at −20° C. After permeabilization in PBST containing fresh 0.1% sodium citrate for 15 min, they were assayed using the in situ cell death detection kit TMR red (Roche) according to the manufacturer's instructions. High-resolution images were obtained by confocal microscope.

### Immunostaining

Embryos were dechorionated and fixed in 4% paraformaldehyde overnight at 4° C. After three washes with PBDT (PBS containing 2% BSA, 1% DMSO and 0.5% Triton X-100), the embryos were incubated with cold acetone for 20 min at −20° C. followed by three additional PBDT washes. Subsequently, the embryos were blocked with 13 blocking buffer (Roche) for 1 h, then incubated with mouse anti-acetylated tubulin monoclonal antibody, 1:200 (Sigma) overnight at 4° C. The embryos were washed extensively in PBDT (30 min, six times) and incubated with Alexa Fluor 568 goat-anti-mouse IgG antibody, 1:200 (Molecular Probe) for 4 h. After five washes in PBDT (30 min each), the embryos were refixed in 4% paraformaldehyde overnight at 4° C.

### Image Acquisition and Microscope Settings

Fluorescent images of the TUNEL assays and the acetylated tubulin staining were obtained with an LSM510 confocal laser scanning microscope (Carl Zeiss Vision GmbH). A bright-field image was acquired at the same time as the fluorescent image. Projection of image stacks was made by the Zeiss image browser. Images were then imported into Adobe Photoshop for cropping, resizing, and orientation. Contrast and brightness were adjusted equally for all images of the same figure.

Images of live embryos were obtained by an SZX12 stereomicroscope (Olympus) and a Magna FIRE SP camera (Olympus). The embryos were mounted in 3% methyl-cellulose. Images were acquired with a 653 objective, at a resolution of 1280×1024, with ˜100-msec exposure and 8-bit depth at room temperature. The image set of each embryo was combined, resized, cropped, and oriented using Adobe Photoshop.

### Statistical Analysis

Two-tail t-tests were used to determine the significance of differences between the treated samples and the controls where values resulted from luciferase reporter assay, qRT-PCR, Western blots, or high content screening. The tests were performed using Microsoft Excel, where the test type is always set to two sample equal variance.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

While the invention has been described with reference to specific methods and embodiments, it will be appreciated that various modifications and changes may be made without departing from the invention.

### REFERENCES

Almog, N. and Rotter, V. 1997. Involvement of p53 in cell differentiation and development. Biochim. Biophys. Acta 1333: F1-F27. doi: 10.1016/S0304-419X(97)00012-7.
Bartel, D. P. 2004. MicroRNAs: Genomics, biogenesis, mechanism, and function. Cell 116: 281-297.
Berghmans, S., Murphey, R. D., Wienholds, E., Neuberg, D., Kutok, J. L., Fletcher, C. D., Morris, J. P., Liu, T. X., Schulte-Merker, S., Kanki, J. P., et al. 2005. tp53 mutant zebrafish develop malignant peripheral nerve sheath tumors. Proc. Natl. Acad. Sci. 102: 407-412.
Bernstein, E., Kim, S. Y., Carmell, M. A., Murchison, E. P., Alcorn, H., Li, M. Z., Mills, A. A., Elledge, S. J., Anderson, K. V., and Hannon, G. J. 2003. Dicer is essential for mouse development. Nat. Genet. 35: 215-217.
Bloomston, M., Frankel, W. L., Petrocca, F., Volinia, S., Alder, H., Hagan, J. P., Liu, C. G., Bhatt, D., Taccioli, C., and Croce, C. M. 2007. MicroRNA expression patterns to differentiate pancreatic adenocarcinoma from normal pancreas and chronic pancreatitis. JAMA 297: 1901-1908.
Bommer, G. T., Gerin, I., Feng, Y., Kaczorowski, A. J., Kuick, R., Love, R. E., Zhai, Y., Giordano, T. J., Qin, Z. S., Moore, B. B., et al. 2007. p53-mediated activation of miRNA34 candidate tumor-suppressor genes. Curr. Biol. 17: 1298-1307.
Bousquet, M., Quelen, C., Rosati, R., Mansat-De Mas, V., La Starza, R., Bastard, C., Lippert, E., Talmant, P., Lafage-Pochitaloff, M., Leroux, D., et al. 2008. Myeloid cell differentiation arrest by miR-125b-1 in myelodysplastic syndrome and acute myeloid leukemia with the t(2;11)(p21; q23) translocation. J. Exp. Med. 205: 2499-2506.
Brand, M., Granato, M., and Nusslein-Volhard, C. 2002. Keeping and raising zebrafish. In Zebrafish: A practical approach. (eds. C. Nusslein-Volhard and R. Dahm), pp. 7-37. Oxford University Press, New York.
Bretaud, S., Allen, C., Ingham, P. W., and Bandmann, O. 2007. p53-dependent neuronal cell death in a DJ-1-deficient zebrafish model of Parkinson's disease. J. Neurochem. 100: 1626-1635.
Campbell, W. A., Yang, H., Zetterberg, H., Baulac, S., Sears, J. A., Liu, T., Wong, S. T., Zhong, T. P., and Xia, W. 2006. Zebrafish lacking Alzheimer presenilin enhancer 2 (Pen-2) demonstrate excessive p53-dependent apoptosis and neuronal loss. J. Neurochem. 96: 1423-1440.
Caygill, E. E. and Johnston, L. A. 2008. Temporal regulation of metamorphic processes in Drosophila by the let-7 and miR-125 heterochronic microRNAs. Curr. Biol. 18: 943-950.
Chan, C. S., Elemento, O., and Tavazoie, S. 2005. Revealing posttranscriptional regulatory elements through network level conservation. PLoS Comput. Biol. 1: e69. doi: 10.1371/journal.pcbi.0010069.
Chang, T. C., Wentzel, E. A., Kent, O. A., Ramachandran, K., Mullendore, M., Lee, K. H., Feldmann, G., Yamakuchi, M., Ferlito, M., Lowenstein, C. J., et al. 2007. Transactivation of miR-34a by p53 broadly influences gene expression and promotes apoptosis. Mol. Cell. 26: 745-752.
Chen, K. and Rajewsky, N. 2006. Deep conservation of micro-RNA-target relationships and 39UTR motifs in vertebrates, flies, and nematodes. Cold Spring Harb. Symp. Quant. Biol. 71: 149-156.
Dony, C., Kessel, M., and Gruss, P. 1985. Post-transcriptional control of myc and p53 expression during differentiation of the embryonal carcinoma cell line F9. Nature 317: 636-639.
Ekker, S. C. and Larson, J. D. 2001. Morphant technology in model developmental systems. Genesis 30: 89-93.
Foshay, K. M. and Gallicano, G. I. 2007. Small RNAs, big potential: The role of MicroRNAs in stem cell function. Curr. Stem Cell Res. Ther. 2: 264-271.
Foulkes, W. D. 2007. p53—Master and commander. N. Engl. J. Med. 357: 2539-2541.
Garzon, R., Fabbri, M., Cimmino, A., Calin, G. A., and Croce, C. M. 2006. MicroRNA expression and function in cancer. Trends Mol. Med. 12: 580-587.
Ghiselli, G. 2006. SMC3 knockdown triggers genomic instability and p53-dependent apoptosis in human and zebrafish cells. Mol. Cancer. 5: 52.
Giraldez, A. J., Cinalli, R. M., Glasner, M. E., Emright, A. J., Thomson, J. M., Baskerville, S., Hammond, S. M., Bartel, D. P., and Schier, A. F. 2005. MicroRNAs regulate brain morphogenesis in zebrafish. Science 308: 833-838.
Harris, C. C. 1996. The 1995 Walter Hubert Lecture—molecular epidemiology of human cancer: Insights from the mutational analysis of the p53 tumour-suppressor gene. Br. J. Cancer 73: 261-269.
He, L. and Hannon, G. J. 2004. MicroRNAs: Small RNAs with a big role in gene regulation. Nat. Rev. Genet. 5: 522-531. He, L., He, X., Lim, L. P., de Stanchina, E., Xuan, Z., Liang, Y.,
Xue, W., Zender, L., Magnus, J., Ridzon, D., et al. 2007. A microRNA component of the p53 tumour suppressor network. Nature 447: 1130-1134.
Iorio, M. V., Visone, R., Di, L. G., Donati, V., Petrocca, F., Casalini, P., Taccioli, C., Volinia, S., Liu, C. G., Alder, H., et al. 2007. MicroRNA signatures in human ovarian cancer. Cancer Res. 67: 8699-8707.
Johnson, S. M., Grosshans, H., Shingara, J., Byrom, M., Jarvis, R., Cheng, A., Labourier, E., Reinert, K. L., Brown, D., and Slack, F. J. 2005. RAS is regulated by the let-7 microRNA family. Cell 120: 635-647.
Khochbin, S. and Lawrence, J. J. 1989. An antisense RNA involved in p53 mRNA maturation in murine erythroleukemia cells induced to differentiate. EMBO J. 8: 4107-4114.
Klinken, S. P., Holmes, K. L., Morse III, H. C., and Thorgeirsson, S. S. 1988. Transcriptional and post-transcriptional regulation of c-myc, c-myb, and p53 during proliferation and differentiation of murine erythroleukemia cells treated with DFMO and DMSO. Exp. Cell Res. 178: 185-198.

Kloosterman, W. P., Lagendijk, A. K., Ketting, R. F., Moulton, J. D., and Plasterk, R. H. 2007. Targeted inhibition of miRNA maturation with morpholinos reveals a role for miR-375 in pancreatic islet development. PLoS Biol. 5: e203. doi: 10.1371/journal.pbio.0050203.
Kuerbitz, S. J., Plunkett, B. S., Walsh, W. V., and Kastan, M. B. 1992. Wild-type p53 is a cell cycle checkpoint determinant following irradiation. Proc. Natl. Acad. Sci. 89: 7491-7495.
Langheinrich, U., Hennen, E., Stott, G., and Vacun, G. 2002. Zebrafish as a model organism for the identification and characterization of drugs and genes affecting p53 signaling. Curr. Biol. 12: 2023-2028.
Lee, K. C., Goh, W. L., Xu, M., Kua, N., Lunny, D., Wong, J. S., Coomber, D., Vojtesek, B., Lane, E. B., and Lane, D. P. 2008. Detection of the p53 response in zebrafish embryos using new monoclonal antibodies. Oncogene 27: 629-640.
Lewis, B. P., Burge, C. B., and Bartel, D. P. 2005. Conserved seed pairing, often flanked by adenosines, indicates that thousands of human genes are microRNA targets. Cell 120: 15-20.
Li, W., Xie, L., He, X., Li, J., Tu, K., Wei, L., Wu, J., Guo, Y., Ma, X., Zhang, P., et al. 2008. Diagnostic and prognostic implications of microRNAs in human hepatocellular carcinoma. Int. J. Cancer 123: 1616-1622.

Moll, U. M., Ostermeyer, A. G., Haladay, R., Winkfield, B., Frazier, M., and Zambetti, G. 1996. Cytoplasmic sequestration of wild-type p53 protein impairs the G1 checkpoint after DNA damage. Mol. Cell. Biol. 16: 1126-1137.
Nam, E. J., Yoon, H., Kim, S. W., Kim, H., Kim, Y. T., Kim, J. H., Kim, J. W., and Kim, S. 2008. MicroRNA expression profiles Le et al. in serous ovarian carcinoma. Clin. Cancer Res. 14: 2690-2695.
Nelson, P. T., Baldwin, D. A., Kloosterman, W. P., Kauppinen, S., Plasterk, R. H., and Mourelatos, Z. 2006. RAKE and LNA-ISH reveal microRNA expression and localization in archival human brain. RNA 12: 187-191.
Olsen, P. H. and Ambros, V. 1999. The lin-4 regulatory RNA controls developmental timing in Caenorhabditis elegans by blocking LIN-14 protein synthesis after the initiation of translation. Dev. Biol. 216: 671-680.
Rajewsky, N. 2006. microRNA target predictions in animals. Nat. Genet. 38: S8-S13. doi: 10.1038/ng1798.
Robu, M. E., Larson, J. D., Nasevicius, A., Beiraghi, S., Brenner, C., Farber, S. A., and Ekker, S. C. 2007. p53 activation by knockdown technologies. PLoS Genet. 3: e78. doi: 10.1371/journal.pgen.0030078.
Ronca, F., Chan, S. L., and Yu, V. C. 1997. 1-(5-Isoquinolinesulfonyl)-2-methylpiperazine induces apoptosis in human neuroblastoma cells, SH-SY5Y, through a p53-dependent pathway. J. Biol. Chem. 272: 4252-4260.
Shi, X. B., Xue, L., Yang, J., Ma, A. H., Zhao, J., Xu, M., Tepper, C. G., Evans, C. P., Kung, H. J., and Vere White, R. W. 2007. An androgen-regulated miRNA suppresses Bak1 expression and induces androgen-independent growth of prostate cancer cells. Proc. Nail. Acad. Sci. 104: 19983-19988.
Sinha, A. U., Kaimal, V., Chen, J., and Jegga, A. G. 2008. Dissecting microregulation of a master regulatory network. BMC Genomics 9: 88. doi: 10.1186/1471-2164-9-88.
Tarasov, V., Jung, P., Verdoodt, B., Lodygin, D., Epanchintsev, A., Menssen, A., Meister, G., and Hermeking, H.2007. Differential regulation of microRNAs by p53 revealed by massively parallel sequencing: miR-34a is a p53 target that induces apoptosis and G1-arrest. Cell Cycle 6: 1586-1593.
Tay, Y. M., Tam, W. L., Ang, Y. S., Gaughwin, P. M., Yang, H., Wang, W., Liu, R., George, J., Ng, H. H., Perera, R. J., et al. 2008. MicroRNA-134 modulates the differentiation of mouse embryonic stem cells, where it causes post-transcriptional attenuation of Nanog and LRH1. Stem Cells 26: 17-29.
Tchang, F., Gusse, M., Soussi, T., and Mechali, M. 1993. Stabilization and expression of high levels of p53 during early development in Xenopus laevis. Dev. Biol. 159: 163-172.
Vogan, K., Bernstein, M., Leclerc, J. M., Brisson, L., Brossard, J., Brodeur, G. M., Pelletier, J., and Gros, P. 1993. Absence of p53 gene mutations in primary neuroblastomas. Cancer Res. 53: 5269-5273.
Volinia, S., Calin, G. A., Liu, C. G., Ambs, S., Cimmino, A., Petrocca, F., Visone, R., Iorio, M., Roldo, C., Ferracin, M., et al. 2006. A microRNA expression signature of human solid tumors defines cancer gene targets. Proc. Natl. Acad. Sci. 103: 2257-2261.
Wienholds, E., Kloosterman, W. P., Miska, E., Alvarez-Saavedra, E., Berezikov, E., de Bruijn, E., Horvitz, H. R., Kauppinen, S., and Plasterk, R. H. 2005. MicroRNA expression in zebrafish embryonic development. Science 309: 310-311.
Yamaguchi, M., Fujimori-Tonou, N., Yoshimura, Y., Kishi, T., Okamoto, H., and Masai, I. 2008. Mutation of DNA primase causes extensive apoptosis of retinal neurons through the activation of DNA damage checkpoint and tumor suppressor p53. Development 135: 1247-1257.

Yoon, S, and De, M. G. 2006. Computational identification of microRNAs and their targets. Birth Defects Res. C Embryo Today 78: 118-128.
Zhang, B., Pan, X., Cobb, G. P., and Anderson, T. A. 2007. micro-RNAs as oncogenes and tumor suppressors. Dev. Biol. 302: 1-12.

1. A method of regulating apoptosis comprising modulation of the level of a micro RNA-125b having a microRNA response element (MRE) comprising SEQ ID NO. 1, by administering a modulator to the cell.

2. The method of claim 1, wherein the cell is in vitro.

3. The method of claim 1, wherein the cell is in vivo, and the modulator is administered to a subject.

4. The method of any one of claims 1 to 3 wherein apoptosis is upregulated by inhibiting the level of the micro RNA-125b in the cell, by administering the modulator to the cell.

5. The method of claim 4, wherein the modulator is a microRNA-125b antisense oligonucleotide.

6. The method of claim 4, wherein the modulator is a microRNA-125b morpholino.

7. The method of claim 7, wherein the morpholino comprises SEQ ID. NO.: 2.

8. The method of claim 7, wherein the morpholino comprises SEQ ID. NO.: 3.

9. The method of claim 7, wherein the morpholino comprises SEQ ID. NO.: 4.

10. The method of claim 7, wherein the morpholino comprises SEQ ID. NO.: 5.

11. The method of claim 7, wherein the morpholino comprises one or more morpholino selected from SEQ ID. NO.: 2; SEQ ID. NO.: 3; SEQ ID. NO.: 4 and SEQ ID. NO.: 5.

12. The method of any one of claims 4 to 11 wherein the modulator is administered to a subject in need of cancer treatment.

13. The method of any one of claims 1 to 3 wherein apoptosis is downregulated by enhancing the level of the micro RNA-125b in the cell, by administering the modulator to the cell.

14. The method of claim 13, wherein the modulator comprises a vector incorporating a micro RNA-125b having a microRNA response element (MRE) comprising SEQ ID NO. 1

15. The method of claim 13 or 14, wherein the modulator is a microRNA-125b duplex molecule.

16. The method of any one of claims 13 to 15 wherein the modulator is administered to an undifferentiated cell to enhance cell differentiation.

17. A composition for use in regulating apoptosis in a cell wherein the composition modulates the level of a micro RNA-125b having a microRNA response element (MRE) comprising SEQ ID NO. 1.

18. The composition of claim 17, wherein apoptosis is upregulated by the composition inhibiting the level of the micro RNA-125b in the cell.

19. The composition of claim 18 comprising a microRNA-125b antisense oligonucleotide.

20. The composition of claim 18 comprising a microRNA-125b morpholino.

21. The composition of claim 20, wherein the morpholino comprises SEQ ID. NO.: 2.

22. The composition of claim 20, wherein the morpholino comprises SEQ ID. NO.: 3.

23. The composition of claim 20, wherein the morpholino comprises SEQ ID. NO.: 4.

24. The composition of claim 20, wherein the morpholino comprises SEQ ID. NO.: 5.

25. The composition of claim 20, wherein the morpholino comprises one or more morpholino selected from SEQ ID. NO.: 2; SEQ ID. NO.: 3; SEQ ID. NO.: 4 and SEQ ID. NO.: 5.

26. The composition of any one of claims 18 to 25 for use in treating cancer.

27. The composition of claim 17, wherein apoptosis is downregulated by the composition enhancing the level of the micro RNA-125b in the cell.

28. The composition of claim 27 comprising a vector incorporating a micro RNA-125b having a microRNA response element (MRE) comprising SEQ ID NO. 1

29. The composition of claim 27 comprising a microRNA-125b duplex molecule.

30. The composition of any one of claims 27 to 29 for use in differentiating an undifferentiated cell.
